SEQUENCE LISTING (54 sequences)

SEQ ID NO: 1 - protein, 126 aa, Homo sapiens
MSIGVPIKVLHEAEGHIVTCETNTGEVYRGKLIEAEDNMNCQMSNITVTYRDGRVAQLEQVYIRGSKIRFLILPDMLKNAPMLKSMKNKNQGSGAGRGKAAILKAQVAARGRGRGMGRGNIFQKRR

SEQ ID NO: 2 - protein, 86 aa, Homo sapiens
MSLPLNPKPFLNGLTGKPVMVKLKWGMEYKGYLVSVDGYMNMQLANTEEYIDGALSGHLGEVLIRCNNVLYIRGVEEEEEDGEMRE

SEQ ID NO: 3 - protein, 231 aa, Homo sapiens
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGLL

SEQ ID NO: 4 - protein, 76 aa, Homo sapiens
MSKAHPPELKKFMDKKLSLKLNGGRHVQGILRGFDPFMNLVIDECVEMATSGQQNNIGMVVIRGNSIIMLEALERV

SEQ ID NO: 5 - protein, 92 aa, Homo sapiens
MAYRGQGQKVQKVMVQPINLIFRYLQNRSRIQVWLYEQVNMRIEGCIIGFDEYMNLVLDDAEEIHSKTKSRKQLGRIMLKGDNITLLQSVSN

SEQ ID NO: 6 - protein, 118 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPR

SEQ ID NO: 7 - protein, 118 aa, Homo sapiens
MSLLNKPKSEMTPEELQKREEEEFNTGPLSVLTQSVKNNTQVLINCRNNKKLLGRVKAFDRHCNMVLENVKEMWTEVPKSGKGKKKSKPVNKDRYISKMFLRGDSVIVVLRNPLIAGK

SEQ ID NO: 8 - protein, 177 aa, synthetic construct
MSLLNKPKSEMTPEELQKREEEEFNTGPLSVLTQSVKNNTQVLINCRNNKKLLGRVKAFDRHCNMVLENVKEMWTEVPKSGKGKKKSKPVNKDRYISKMFLRGDSVIVVLRNPLIAGKGGSGGSNSDSECPLSHDGYCLHDGVCMYIEALDKYACNCVVGYIGERCQYRDLKWWELR

SEQ ID NO: 9 - protein, 240 aa, synthetic construct
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGPPPPGMRPPRP

SEQ ID NO: 10 - protein, 118 aa, synthetic construct
SKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPR

SEQ ID NO: 11 - protein, 133 aa, Homo sapiens
MNYMPGTASLIEDIDKKHLVLLRDGRTLIGFLRSIDQFANLVLHQTVERIHVGKKYGDIPRGIFVVRGENVVLLGEIDLEKESDTPLQQVSIEEILEEQRVEQQTKLEAEKLKVQALKDRGLSIPRADTLDEY

SEQ ID NO: 12 - protein, 95 aa, Homo sapiens
MLFYSFFKSLVGKDVVVELKNDLSICGTLHSVDQYLNIKLTDISVTDPEKYPHMLSVKNCFIRGSVVRYVQLPADEVDTQLLQDAARKEALQQKQ

SEQ ID NO: 13 - protein, 102 aa, Homo sapiens
MADDVDQQQTTNTVEEPLDLIRLSLDERIYVKMRNDRELRGRLHAYDQHLNMILGDVEETVTTIEIDEETYEEIYKSTKRNIPMLFVRGDGVVLVAPPLRVG

SEQ ID NO: 14 - protein, 139 aa, Homo sapiens
MLPLSLLKTAQNHPMLVELKNGETYNGHLVSCDNWMNINLREVICTSRDGDKFWRMPECYIRGSTIKYLRIPDEIIDMVKEEVVAKGRGRGGLQQQKQQKGRGMGGAGRGVFGGRGRGGIPGTGRGQPEKKPGRQAGKQ

SEQ ID NO: 15 - protein, 91 aa, Homo sapiens
MAANATTNPSQLLPLELVDKCIGSRIHIVMKSDKEIVGTLLGFDDFVNMVLEDVTEFEITPEGRRITKLDQILLNGNNITMLVPGGEGPEV

SEQ ID NO: 16 - protein, 80 aa, Homo sapiens
MSLRKQTPSDFLKQIIGRPVVVKLNSGVDYRGVLACLDGYMNIALEQTEEYVNGQLKNKYGDAFIRGNNVLYISTQKRRM

SEQ ID NO: 17 - protein, 103 aa, Homo sapiens
MADKEKKKKESILDLSKYIDKTIRVKFQGGREASGILKGFDPLLNLVLDGTIEYMRDPDDQYKLTEDTRQLGLVVCRGTSVVLICPQDGMEAIPNPFIQQQDA

SEQ ID NO: 18 - protein, 96 aa, Homo sapiens
MTSALENYINRTVAVITSDGRMIVGTLKGFDQTINLILDESHERVFSSSQGVEQVVLGLYIVRGDNVAVIGEIDEETDSALDLGNIRAEPLNSVAH

SEQ ID NO: 19 - protein, 123 aa, Homo sapiens
MAVSHSVKERTISENSLIILLQGLQGRVTTVDLRDESVAHGRIDNVDAFMNIRLAKVTYTDRWGHQVKLDDLFVTGRNVRYVHIPDDVNITSTIEQQLQIIHRVRNFGGKGQGRWEFPPKNCK

SEQ ID NO: 20 - protein, 360 aa, Homo sapiens
MEERERGARSAGAGSPARPPSPRLDVSSDSFDPLLALYAPRLPPIPYPNAPCFNNVAEYESFLRTGVRGGGRGRGRARGAAAGSGVPAAPGPSGRTRRRPDAPAPDPERIQRLRRLMVAKEEGDGAAGAGRRGPGRSRKAPRNVLTRMPLHEGSPLGELHRCIREGVKVNVHIRTFKGLRGVCTGFLVAFDKFWNMALTDVDETYRKPVLGKAYERDSSLTLTRLFDRLKLQDSSKKEADSKSAVEDSTLSRYSQTSTWKLASVWGRADTGRGSHKRSRSVPSSLQASAREESRSELSGRTTRTDGSSVGGTFSRATTLSRGQSRKKKRKPKVDYQQVFTRHINQIFIRGENVLLVHLAQ

SEQ ID NO: 21-26 - skipped (6 entries)

SEQ ID NO: 27 - RNA, 58 nt, synthetic construct
  source 1..58: mol_type = other RNA
caatttttgaccttgacgatacagctaattcctcgaggaattagctgtatcgtcaagg

SEQ ID NO: 28 - RNA, 58 nt, synthetic construct
  source 1..58: mol_type = other RNA
caatttttgagcaagctgaccctgaagttcactcgagtgaacttcagggtcagcttgc

SEQ ID NO: 29 - DNA, 22 bp, synthetic construct
  source 1..22: mol_type = other DNA
  misc_feature 1..22: note = KRAS 1 forward primer
tggggagggctttctttgtgta

SEQ ID NO: 30 - DNA, 20 bp, synthetic construct
  source 1..20: mol_type = other DNA
  misc_feature 1..20: note = KRAS 1 reverse primer
tgctaagtcctgagcctgtt

SEQ ID NO: 31 - DNA, 20 bp, synthetic construct
  source 1..20: mol_type = other DNA
  misc_feature 1..20: note = KRAS 2 forward primer
ggcaagagtgccttgacgat

SEQ ID NO: 32 - DNA, 20 bp, synthetic construct
  source 1..20: mol_type = other DNA
  misc_feature 1..20: note = KRAS 2 reverse primer
ctcctcttgacctgctgtgt

SEQ ID NO: 33 - DNA, 19 bp, synthetic construct
  source 1..19: mol_type = other DNA
gaaggtgaaggtcggagtc

SEQ ID NO: 34 - DNA, 20 bp, synthetic construct
  source 1..20: mol_type = other DNA
gaagatggtgatgggatttc

SEQ ID NO: 35 - RNA, 144 nt, Homo sapiens
  source 1..144: mol_type = other RNA
agctttgcgcagtggcagtatcgtagccaatgaggtctatccgaggcgcgattattgctaattgaaaacttttcccaataccccgccgtgacgacttgcaatatagtcggcactggcaatttttgacagtctctacggagactg

SEQ ID NO: 36 - RNA, 60 nt, synthetic construct
  source 1..60: mol_type = other RNA
caatttttgaccttgacgatacagctaattcctcgaggaattagctgtatcgtcaaggct

SEQ ID NO: 37 - RNA, 24 nt, synthetic construct
  source 1..24: mol_type = other RNA
ccttgacgatacagctaattcaga

SEQ ID NO: 38 - RNA, 36 nt, synthetic construct
  source 1..36: mol_type = other RNA
caatttttgatctgaattagctgtatcgtcaaggtt

SEQ ID NO: 39 - protein, 158 aa, synthetic construct
MGPETLCGAELVDALQFVCGDRGFYFNKPTGYGSSSRRAPQTGIVDECCFRSCDLRRLEMYCAPLKPAKSATSSATAGGSGGSSKAHPPELKKFMDKKLSLKLNGGRHVQGILRGFDPFMNLVIDECVEMATSGQQNNIGMVVIRGNSIIMLEALERV

SEQ ID NO: 40 - protein, 70 aa, synthetic construct
GPETLCGAELVDALQFVCGDRGFYFNKPTGYGSSSRRAPQTGIVDECCFRSCDLRRLEMYCAPLKPAKSA

SEQ ID NO: 41 - protein, 109 aa, synthetic construct
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVLAEALAEALEALAA

SEQ ID NO: 42 - protein, 95 aa, synthetic construct
MHEHEHEHEHSLPLNPKPFLNGLTGKPVMVKLKWGMEYKGYLVSVDGYMNMQLANTEEYIDGALSGHLGEVLIRCNNVLYIRGVEEEEEDGEMRE

SEQ ID NO: 43 - protein, 9 aa, synthetic construct
HEHEHEHEH

SEQ ID NO: 44 - protein, 14 aa, synthetic construct
LAEALAEALEALAA

SEQ ID NO: 45 - RNA, 35 nt, Human coronavirus 229E
  source 1..35: mol_type = unassigned RNA
caatttttgagttgaattgcttaagcagtatactt

SEQ ID NO: 46 - RNA, 27 nt, Human coronavirus 229E
  source 1..27: mol_type = unassigned RNA
aagtatactgcttaagcaattcaacac

SEQ ID NO: 47 - RNA, 35 nt, Human coronavirus 229E
  source 1..35: mol_type = unassigned RNA
caatttttgagtggtgcttatagtgatactactag

SEQ ID NO: 48 - RNA, 27 nt, Human coronavirus 229E
  source 1..27: mol_type = unassigned RNA
ctagtagtatcactataagcaccacac

SEQ ID NO: 49 - DNA, 21 bp, synthetic construct
  source 1..21: mol_type = other DNA
  misc_feature 1..21: note = SPIKE forward primer
acctatcgtagttgattgctc

SEQ ID NO: 50 - DNA, 20 bp, synthetic construct
  source 1..20: mol_type = other DNA
  misc_feature 1..20: note = SPIKE reverse primer
agcatctcactaacatctgc

SEQ ID NO: 51 - DNA, 25 bp, synthetic construct
  source 1..25: mol_type = other DNA
  misc_feature 1..25: note = ENV forward primer
tggttgttaatgtactactctggtg

SEQ ID NO: 52 - DNA, 23 bp, synthetic construct
  source 1..23: mol_type = other DNA
  misc_feature 1..23: note = ENV reverse primer
acatatggcaagtgaaacaaagc

SEQ ID NO: 53 - DNA, 20 bp, synthetic construct
  source 1..20: mol_type = other DNA
  misc_feature 1..20: note = ACTB forward primer
actcttccagccttccttcc

SEQ ID NO: 54 - DNA, 20 bp, synthetic construct
  source 1..20: mol_type = other DNA
  misc_feature 1..20: note = ACTB reverse primer
cgtacaggtctttgcggatg

(19) United States
(12) Patent Application Publication
(10) Pub. No.: US 2023/0063922 A1
(43) Pub. Date: Mar. 2, 2023

(54) RIBONUCLEOPROTEINS FOR RNA THERAPEUTICS DELIVERY AND GENE SILENCING

(71) Applicant: Hong Kong Baptist University, Hong Kong (CN)

(72) Inventor: Wei Shen AIK, Teluk Intan (MY)

(21) Appl. No.: 17/930,088

(22) Filed: Sep. 7, 2022

Related U.S. Application Data
(63) Continuation-in-part of application No. PCT/CN2021/080443, filed on Mar. 12, 2021.
(60) Provisional application No. 63/242,013, filed on Sep. 8, 2021.
(60) Provisional application No. 62/988,929, filed on Mar. 13, 2020.

(51) Int. Cl. A61K 47/64; C12N 15/113; A61P 35/00; A61P 31/14

(52) U.S. Cl. CPC A61K 47/64; C12N 15/1135; C12N 15/1136; C12N 15/1131; A61P 35/00; A61P 31/14; C12N 2310/531; C12N 2310/14; C12N 2310/3517; C12N 2310/3513; C12N 2320/32

(57) ABSTRACT

The present invention serves as a platform technology to deliver RNA therapeutics into cells. It provides a system for delivery of RNA molecules for biomedical purposes. The modular protein-based system described in this invention allows for customization of protein modules to achieve specificity in cell-targeting, thus having the ability to be optimized for treating different diseases. Examples of types of diseases that could adopt this technology for treatment include cancer, neurodegenerative diseases and viral infection.

### CROSS REFERENCE OF RELATED APPLICATION

This application claims priority from (1) U.S. Provisional Patent Application Ser. No. 63/242,013 filed on Sep. 8, 2021, and is a continuation-in-part under 35 U.S.C. 111(a) of (2) International Patent Application Number PCT/CN2021/080443 filed on Mar. 12, 2021 with a priority claim from U.S. Provisional Patent Application Ser. No. 62/988,929 filed on Mar. 13, 2020, which are hereby incorporated by reference in their entirety for all purposes.

### REFERENCE TO SEQUENCE DISCLOSURE

The sequence listing file under the file name “Sequence Listing 034590-000026.xml” submitted in ST.26 XML file format with a file size of 50 KB on Sep. 7, 2022 is incorporated herein by reference.

### FIELD OF THE INVENTION

The present invention relates to ribonucleoproteins for RNA therapeutic delivery and gene silencing. In particular, the present invention provides RNA therapeutic delivery agents using modified ribonucleoprotein complexes as the delivery system for gene therapy to treat a variety of diseases and incorporating endosomal escape peptides (EEP) to facilitate the delivered payload to silence desired genes.

### BACKGROUND OF THE INVENTION

RNA interference (RNAi) is a gene silencing process initiated by double-stranded RNA (dsRNA) in the cell. RNAi plays an important role in cellular defense against viruses, which often generate dsRNAs during replication. The discovery of RNAi led to the 2006 Nobel Prize in Physiology and Medicine shared by Craig Mello and Andrew Fire. One of the main pathways of RNAi involves small interfering RNA (siRNA), a short -21 nucleotide dsRNA. siRNA is generated by Dicer from longer dsRNAs (e.g. shRNA). siRNA is then unwound into single-stranded RNAs, with one of the strands being loaded onto the RNA-induced silencing complex (RISC) to guide degradation of target RNAs through sequence complementarity.

The ability of RNAi to silence specific gene has prompted researchers to explore its therapeutic potential in treating cancer, viral infection, and neurodegenerative diseases. However, siRNA delivery remains challenging. To effectively deliver siRNA therapeutics, several strategies have been devised to protect and stabilize siRNA and carry it into cells. The most effective method to date uses lipid-based carriers to encapsulate or bind siRNA, and carry it across the plasma membrane. This strategy has shown promise resulting in clinical trials to treat hepatitis B, pancreatic cancer, hypercholesterolemia etc. However, there are some drawbacks, including toxicity and potential to elicit an immune response. To protect siRNA from nuclease degradation, chemical modifications have been added to siRNAs, e.g. 2′O-methylation and 2′O-methyl phosphorodithioate. Yet, there have been reports that chemical modifications could reduce siRNA effectiveness. Viral vectors have also been engineered as siRNA delivery agents because viruses are effective gene-delivery vehicles. Despite their potential, viral vectors carry a high risk in triggering an immune response.

Other proposed methods include siRNA conjugation (e.g. PEG, aptamer, cholesterol, Gal-NAc), and using exosomes, inorganic materials, and proteins as carriers. In particular, N-acetogalactosamine (GalNAc), which binds to liver cell receptors, has been successfully used as liver-targeting ligand conjugate. Alnylam Pharmaceuticals, which developed Patisiran, pioneered the use of GalNAc conjugate for siRNA delivery. Owing to this, liver siRNA delivery technology has made significant advances relative to delivery to other organs. Effective delivery of RNA therapeutics to organs other than liver appears to be a bottleneck for RNA therapy.

### SUMMARY OF THE INVENTION

Accordingly, it is an objective of the present invention to provide an RNA therapeutic delivery agent using RNA binding proteins as the delivery system for gene therapy to treat a variety of diseases.

In a first aspect, there is provided an RNA therapeutic delivery agent including:
a modified small nuclear ribonucleoprotein (snRNP) complex for delivering a therapeutic RNA to a biological cell, where the modified snRNP complex comprises a core, and where the core comprises:
one or more RNA molecules;
one or more Sm proteins, or one or more LSm proteins, or any combination of Sm and LSm proteins, or any variants thereof; and
a Sm binding sequence,
wherein the Sm binding sequence is attached to a therapeutic RNA, and wherein the therapeutic RNA is bound to at least one of the Sm proteins or at least one of the LSm proteins, or any combination or variant thereof, of the modified snRNP complex,
wherein at least one cell receptor ligand is attached to at least one of the Sm proteins or at least one of the LSm proteins, or any combination or variant thereof, of the modified snRNP complex, and
wherein at least one endosomal escape peptide is attached to at least one of the Sm proteins or at least one of the LSm proteins, or any combination or variants thereof, of the modified snRNP complex.

In a first embodiment of the first aspect, the one or more Sm proteins comprise SmD3, SmF, SmB, SmG, SmE, SmD1 and SmD2 of SEQ ID NOs: 1-7, respectively, or the variants thereof comprising SmB′ of SEQ ID NO: 9, SmD1′ of SEQ ID NO: 10, IGF-SmG of SEQ ID NO: 39, SmB₁₋₉₅-GALAS of SEQ ID NO: 41, and H5E-SmF of SEQ ID NO: 42.

In a second embodiment of the first aspect, the LSm proteins comprise LSm1, LSm2, LSm3, LSm4, LSm5, LSm6, LSm7, LSm8, LSm10, and LSm11 of SEQ ID NOs: 11-20, respectively.

In a third embodiment of the first aspect, the cell receptor ligand is for receptor-mediated endocytosis comprising epidermal growth factor (EGF) and any family members thereof, and wherein the EGF or any family members thereof is/are attached to any of the Sm proteins, or their variants, for example, SmD2.

In a further embodiment of the first aspect, the cell receptor ligand further comprises insulin-like growth factor (IGF), wherein the IGF or any family members thereof is/are attached to any of the Sm proteins, or their variants, for example, SmG.

In certain embodiments, the endosomal escape peptides include, but not limited to, H5E and GALAS.

In a fourth embodiment of the first aspect, the therapeutic RNA is incorporated into the short-hairpin ribonucleoprotein complex (shRNP) comprising an shRNA of SEQ ID NO: 27 or 36 attached with a 6-FAM fluorescent label at 5′-end thereof for targeting KRAS, an shRNA of SEQ ID NO: 28 attached with a DY547 dye at 5′-end thereof for targeting egfp, or a small-interfering RNA (siRNA) of SEQ ID NO: 37 and 38 for targeting KRAS, a pair of siRNAs of SEQ ID Nos: 45 and 46 for targeting a spike protein of a coronavirus, or a pair of siRNAs of SEQ ID Nos: 47 and 48 for targeting an envelope protein of the coronavirus.

In certain embodiments, other siRNAs targeting various viruses and/or cancers can also be incorporated into said shRNP for gene silencing.

In a fifth embodiment of the first aspect, the Sm binding sequence is attached to the RNA at either 3′-end or 5′-end thereof.

In a sixth embodiment of the first aspect, the cell receptor ligand is attached to N-terminus, C-terminus, or within a loop between strands 3 and 4 of any one of the Sm proteins or any one of the LSm proteins, or any variant thereof.

In a seventh embodiment of the first aspect, the one or more RNA molecules comprise small nuclear RNA.

In an eighth embodiment of the first aspect, the Sm binding sequence is one of SEQ ID Nos: 21-26, where SEQ ID NO: 21 is 5′-AAUUUGUGG-3′; SEQ ID NO: 22 is 5′-GAUUUUUGG-3′; SEQ ID NO: 23 is 5′-AAUUUUUGA-3′; SEQ ID NO: 24 is 5′-AAUUUUUUG-3′; SEQ ID NO: 25 is 5′-UUUU-3′; SEQ ID NO: 26 is 5′-AAUUUGUCUAG-3′.

In a second aspect of the present invention, there is provided a modified small nuclear ribonucleoprotein (snRNP) complex for gene silencing in a cell, where the snRNP complex comprises a core, and where the core comprises:
one or more RNA molecules;
one or more Sm proteins, or one or more LSm proteins, or any combination of the Sm and LSm proteins, or any variants thereof; and
a Sm binding sequence,
wherein the Sm binding sequence is attached to a therapeutic RNA including shRNA or siRNA to be delivered to the cell,
wherein the shRNA or siRNA is bound to at least one of the Sm proteins or at least one of the LSm proteins, or any combination or variant thereof, of the modified snRNP complex,
wherein at least one cell receptor ligand is attached to at least one of the Sm proteins or at least one of the LSm proteins, or any combination or variant thereof, of the modified snRNP complex, and
wherein at least one endosomal escape peptide is attached to at least one of the Sm proteins or at least one of the LSm proteins, or any combination or variants thereof, of the modified snRNP complex.

In a first embodiment of the second aspect, the one or more Sm proteins comprise SmD3, SmF, SmB, SmG, SmE, SmD1 and SmD2 of SEQ ID Nos: 1-7, respectively, or the variant thereof comprising SmB′ of SEQ ID NO: 9, SmD1′ of SEQ ID NO: 10, IGF-SmG of SEQ ID NO: 39, SmB₁₋₉₅-GALA3 of SEQ ID NO: 41, and H5E-SmF of SEQ ID NO: 42.

In a second embodiment of the second aspect, the LSm proteins comprise LSm1, LSm2, LSm3, LSm4, LSm5, LSm6, LSm7, LSm8, LSm10, and LSm11 of SEQ ID Nos: 11-20, respectively.

In a third embodiment of the second aspect, the at least one cell receptor ligand comprises an epidermal growth factor (EGF) and any family members thereof, and wherein the EGF or any family members thereof is/are attached to any of the Sm proteins, or their variants, for example, SmD2.

In a further embodiment of the second aspect, the cell receptor ligand further comprises IGF, wherein the IGF or any family members thereof is/are attached to any of the Sm proteins, or their variants, for example, SmG.

In certain embodiments, the endosomal escape peptides include, but not limited to, H5E and GALAS.

In a fourth embodiment of the second aspect, the shRNP comprises an shRNA of SEQ ID NO: 27 or 36 attached with a 6-FAM fluorescent label at 5′-end thereof for targeting KRAS, an shRNA of SEQ ID NO: 28 attached with a DY547 dye at 5′-end thereof for targeting egfp, an siRNA of SEQ ID NOs: 37 and 38 for targeting KRAS, a pair of siRNAs of SEQ ID NOs: 45 and 46 for targeting a spike protein of a coronavirus, or a pair of siRNAs of SEQ ID NOs: 47 and 48 for targeting an envelope protein of the coronavirus.

In certain embodiments, other siRNAs targeting various viruses and/or cancers can also be incorporated into said shRNP for gene silencing.

In a fifth embodiment of the second aspect, the Sm binding sequence is attached to the shRNA or siRNA at either 3′-end or 5′-end thereof.

In a sixth embodiment of the second aspect, the cell receptor ligand is attached to N-terminus, C-terminus, or within a loop between strands β and 4 of any one of the Sm proteins or any one of the Sm proteins, or any variant thereof.

In a seventh embodiment of the second aspect, the one or more RNA molecules comprise small nuclear RNA.

In an eighth embodiment of the second aspect, the Sm binding sequence is one of SEQ ID NOs: 21-26, where SEQ ID NO: 21 is 5′-AAUUUGUGG-3′; SEQ ID NO: 22 is 5′-GAUUUUUGG-3′; SEQ ID NO: 23 is 5′-AAUUUUUGA-3′; SEQ ID NO: 24 is 5′-AAUUUUUUG-3′; SEQ ID NO: 25 is 5′-UUUU-3′; SEQ ID NO: 26 is 5′-AAUUUGUCUAG-3′.

Other aspects of the present invention include providing a gene therapy to treat diseases in a subject comprising using the agent or complex or alike according to the first or second aspect of the present invention or described herein.

In certain embodiments, the diseases include, but not limited to, cancers, neurodegenerative disease, and viral infections.

In any aspects of the present invention, the subject receiving the gene therapy according to the method described herein includes human or other animals.

In an exemplary embodiment, the agent or complex described herein is configured to deliver one or more therapeutic RNAs via a non-viral/non-lipid-based transfection method.

Preferably, the one or more therapeutic RNAs is/are delivered to a target cell or tissue of the subject via a protein-based delivery system including the agent or complex described herein, wherein the modified snRNP complex is formed ex vivo with the one or more RNA molecules, the one or more therapeutic RNAs in the presence of the corresponding Sm binding sequences, and one or more cell binding ligands before being delivered to the subject.

In certain embodiments, one or more endosomal escape peptides (EEPs) is further attached to one or more Sm/LSm proteins, or any combination or variants thereof, of the modified snRNP complex before being delivered to the subject.

In certain embodiments, the agent or complex is administered to the subject via one or more of intratumoral, intramuscular, intraperitoneal, and intravenous injections in the absence of any viral/lipid-based vector/carrier.

Those skilled in the art will appreciate that the invention described herein is susceptible to variations and modifications other than those specifically described.

The present invention includes all such variation and modifications. The invention also includes all the steps and features referred to or indicated in the specification, individually or collectively, and any and all combination or any two or more of the steps or features.

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the present invention and apply throughout. Unless otherwise defined, all other technical terms used herein have the same meaning as commonly understood to one of ordinary skill in the art to which the invention belongs.

Other aspects and advantages of the present invention will be apparent to those skilled in the art from a review of the ensuing description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects and features of the present invention will become apparent from the following description of the invention, when taken in conjunction with the accompanying drawings, in which:

FIG. 1 shows hurdles encountered by RNAi therapy and overview of shRNA delivery and RNA interference.

FIG. 2A shows partial model of crystal structure of the U4 snRNP core.

FIG. 2B shows design drawings of short-hairpin ribonucleoprotein complex, shRNP. The N-terminus, C-terminus and the insert between β-strands 3 and 4 of each Sm protein allows for ligand fusion and protein modifications for customisation.

FIG. 3 shows the size exclusion chromatography (SUPERDEX® 200 Increase) and SDS-PAGE analysis of shRNP targeting KRAS. UV absorbance at 280 nm (smooth line), UV absorbance at 254 nm (dotted line), fractions corresponding to peak P1 was analysed by SDS-PAGE and collected.

FIG. 4 shows confocal microscopy images of cellular uptake of shRNP by A549 cells. A549 cells incubated with shRNP complex (bottom), or protein component only (top). Signals from nuclear staining with Hoerscht 33342 (left); signals from the FAM-labeled shRNA (middle); merged signals from nuclear staining and FAM-labeled shRNA (right); scale bar 100 μm.

FIG. 5 shows background fluorescence signals of A549 cells. A549 cells incubated with buffer control (top row), protein components only (middle row), or shRNA only (bottom row). Signals from nuclear staining with Hoerscht 33342 (left column); Signals from background fluorescence using FAM channel (middle column); merged signals from nuclear staining and FAM channels (right column). Fluorescence background speckled signals were present in all three controls but none of them indicated that they were diffused in the cell cytoplasm. Scale bar 100 μm.

FIG. 6A shows the quantitative PCR of KRAS expression for Primer pair 1.

FIG. 6B shows the quantitative PCR of KRAS expression for Primer pair 2.

FIG. 7 shows the cell viability in response to various treatments by MTT assay for A549 cells.

FIG. 8 shows size exclusion chromatography (SUPERDEX® 200 Increase) and SDS-PAGE analysis of shRNP targeting egfp. UV absorbance at 280 nm (smooth line), UV absorbance at 254 nm (dotted line), fractions corresponding to peak P1 was analysed by SDS-PAGE and collected.

FIG. 9 shows eGFP signal reduction in the eGFP-expressing HEK293T cells when shRNP was uptaken. The eGFP-expressing HEK293T cells were incubated with buffer control (top row), protein components used to assemble shRNP (second row), shRNA only (third row), or shRNP complex targeting GFP (bottom row). shRNA is tagged with DY547 dye on the 5′-end. Signals from nuclear staining with Hoerscht 33342 (first column); signals from eGFP (second column); signals from DY547 dye or shRNA (third column); merged signals from nuclear staining, eGFP and DY547 dye (fourth column). Representative cells that show RNAi effect from shRNP entry contain DY547-shRNA signals and reduced eGFP signal are indicated with arrows (fourth row); scale bar 10 μm.

FIG. 10 shows the size exclusion chromatography (SUPERDEX® 200 Increase) and SDS-PAGE analysis of a 7-protein shRNP targeting KRAS. UV absorbance at 280 nm (smooth line), UV absorbance at 260 nm (dotted line), fractions corresponding to peak P1 was analysed by SDS-PAGE and collected.

FIG. 11 shows the size exclusion chromatography (SUPERDEX® 200 Increase) and SDS-PAGE analysis of a 7-protein siRNP targeting KRAS. UV absorbance at 280 nm (smooth line), UV absorbance at 260 nm (dotted line), fractions corresponding to peak P1 was analysed by SDS-PAGE and collected.

FIG. 12 shows the quantitative PCR of KRAS expression of A549 cells when tested with shRNP or siRNP. Error bars indicate standard deviation of the mean from duplicates of quantitative PCR reactions (n=2).

FIG. 13 shows the size exclusion chromatography (SUPERDEX® 200 Increase) and SDS-PAGE analysis of shRNP containing endosomal escape peptides GALA3 and H5E and with the shRNA targeting KRAS. UV absorbance at 280 nm (smooth line), UV absorbance at 260 nm (dotted line), fractions corresponding to peak P1 and peak P2 were analysed by SDS-PAGE and collected.

FIG. 14 shows the size exclusion chromatography (SUPERDEX® 200 Increase) and SDS-PAGE analysis of shRNP containing endosomal escape peptides H5E and with the shRNA targeting KRAS. UV absorbance at 280 nm (smooth line), UV absorbance at 260 nm (dotted line), fractions corresponding to peak P1 and peak P2 were analysed by SDS-PAGE and collected.

FIG. 15 shows the quantitative PCR of KRAS expression in A549 cells when tested with shRNP containing endosomal escape peptides. Error bars indicate standard deviation of the mean from duplicates of quantitative PCR reactions (n=2).

FIG. 16 shows confocal microscopy images of cellular uptake of shRNP containing endosomal escape peptides GALA3 and H5E by A549 cells. A549 cells incubated with shRNP complex (bottom), or protein component only (top). Signals from nuclear staining with Hoerscht 33342 (left); signals from the FAM-labeled shRNA (middle); merged signals from nuclear staining and FAM-labeled shRNA (right); scale bar 20 μm.

FIG. 17 shows the quantitative PCR of KRAS expression in A549 cells when tested with siRNPs containing the H5E endosomal escape peptide. Error bars indicate standard deviation of the mean from duplicates of quantitative PCR reactions (n=2).

FIG. 18 shows the fluorescence levels by flow cytometry of A549 cells when tested with shRNP containing different receptor binding ligands.

FIG. 19 shows the quantitative PCR of KRAS expression in HCT116 cells when tested with siRNPs containing the H5E endosomal escape peptide, IGF or EGF ligand, and 4 or 7 Sm proteins. Error bars indicate standard deviation of the mean from duplicates of quantitative PCR reactions (n=2).

FIG. 20 shows the quantitative PCR of KRAS expression in HCT116 cells when tested with different concentrations of siRNP-EGF-H5E. Error bars indicate standard deviation of the mean from duplicates of quantitative PCR reactions (n=2).

FIG. 21 shows the cell viability of HCT116 by MTT assay in response to treatments by different concentrations of siRNP-EGF-H5E. Error bars indicate standard deviation of the mean from duplicate assays (n=2).

FIG. 22 shows the quantitative PCR of SPIKE (5) expression in infected and uninfected VERO cells by HCOV-229E when tested with si(S)RNP-IGF-H5E.

FIG. 23 shows the quantitative PCR of ENVELOPE (E) expression in infected and uninfected VERO cells by HCOV-229E when tested with si(E)RNP-IGF-H5E.

### DETAILED DESCRIPTION OF THE INVENTION

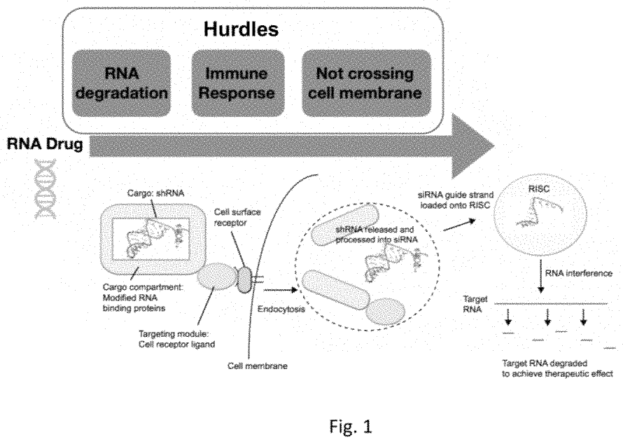

The present invention serves as a platform technology to deliver RNA therapeutics into cells (FIG. 1). As RNA therapeutics has immense potential applications in biotechnology, for example gene silencing for chronic disease and cancer treatment, RNA-based inhibitors as well as RNA-based vaccines, this invention provides a system for delivery of RNA molecules for biomedical purposes. The modular protein-based system described in this invention allows for customization of protein modules to achieve specificity in cell-targeting, thus having the ability to be optimized for treating different diseases. Examples of types of diseases that could adopt this technology for treatment include cancer, neurodegenerative diseases and viral infection.

This invention could also be applied to RNA delivery for research purposes, be used in cell lines and in vivo studies.

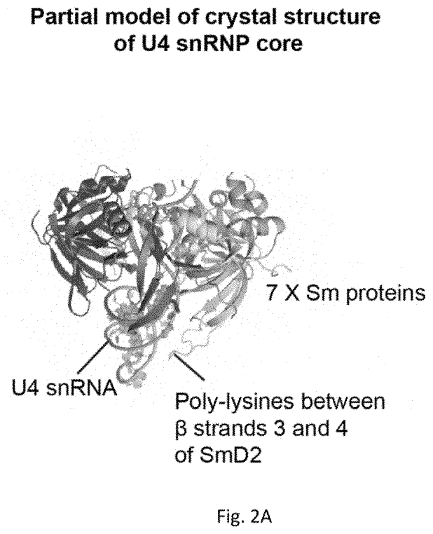

In this invention, a modular RNA delivery agent is constructed using modified components of small nuclear ribonucleoprotein (snRNP) complex. In one embodiment, U4 snRNP is modified to become an RNA delivery agent. U4 snRNP is one of the many snRNPs which can be found in eukaryotic organisms; examples of other snRNPs, which can be other embodiments of the present invention, include but not limited to, U1 snRNP, U2 snRNP, U5 snRNP, U6 snRNP, U7 snRNP, Lsm1-7 ring etc. U4 snRNP is part of spliceosome that splices pre-messenger RNAs. The present snRNP complex includes a core which is comprised of Sm or LSm proteins, of one or more RNA molecules where the RNA molecules contain a Sm binding sequence and the rest of the RNA can be fully or partially double-stranded and either wild-type or modified RNA. For example, in the U4 snRNP core, it can be composed of a 144-nucleotide long U4 small nuclear RNA (snRNA) (SEQ ID NO: 35) and 7 Sm proteins, namely SmD1 (SEQ ID NO: 6), SmD2 (SEQ ID NO: 7), SmG (SEQ ID NO: 4), SmE (SEQ ID NO: 5), SmF (SEQ ID NO: 2), SmD3 (SEQ ID NO: 1) and SmB (SEQ ID NO: 3) or their variant including SmB′ (SEQ ID NO: 9) and SmD1′ (SEQ ID NO: 10). Other snRNPs comprise of different combinations of Sm and like-Sm (LSm) proteins. All Sm and LSm proteins are from the same Sm-fold family, having similar structures and form oligomeric ring structures. In another embodiment, U7 snRNP is composed of LSm11 (SEQ ID NO: 20), LSm10 (SEQ ID NO: 19), SmG, SmE, SmF, SmD3 and SmB or SmB′; U6 snRNP is composed of LSm2 (SEQ ID NO: 12), LSm3 (SEQ ID NO: 13), LSm4 (SEQ ID NO: 14), LSm5 (SEQ ID NO: 15), LSm6 (SEQ ID NO: 16), LSm7 (SEQ ID NO: 17) and LSm8 (SEQ ID NO: 18); LSm1-7 ring is composed of LSm1 (SEQ ID NO: 11), LSm2, LSm3, LSm4, LSm5, LSm6, and LSm7. The Sm proteins for U4 snRNP form a doughnut-shaped ring around the Sm binding site on the U4 snRNA, as revealed by crystal structure (FIG. 2A). To enable the one or more RNA molecules to form a complex with the Sm proteins of snRNP, the corresponding Sm binding sequence is attached to the RNA. Possible points for such attachments are for example, but not limited to, 3′-end or 5′-end of the RNA. As an embodiment, the Sm binding sequence for U4 snRNP (5′-AAUUUUUGA-3′) (SEQ ID NO: 23) is attached to the RNA in order for the RNA to bind to the modified U4 snRNP proteins. Other snRNPs will have variations of Sm binding sequence and variations of Sm proteins, e.g., the Sm binding sequences for UI snRNP (5′-AAUUUGUGG-3′) (SEQ ID NO: 21), for U2 snRNP (5′-GAUUUUUGG-3′) (SEQ ID NO: 22), for U5 snRNP (5′-AAUUUUUUG-3′) (SEQ ID NO: 24), for U6 snRNP (5′-UUUU-3′) (SEQ ID NO: 25), for U7 snRNP (5′-AAUUUGUCUAG-3′) (SEQ ID NO: 26). For cellular uptake via endocytosis, a cell receptor ligand is attached on one of the Sm proteins. As an example, epidermal growth factor (EGF) is attached to SmD2. EGF binds to EGF receptor (EGFR) on the cell surface for endocytic uptake of the RNA delivery agent. Attachment of other ligands for receptor-mediated endocytosis are other embodiments of the present invention, including but not limited to, attaching other members of epidermal growth factor family, e.g., neuregulin-1, neuregulin-2, neuregulin-3, neuregulin-4, amphiregulin, epiregulin, epigen, betacellulin, transforming growth factor-α etc., members of the insulin-like growth factor (IGF) family, e.g. IGF-I and IGF-II etc., and neuropeptide families, e.g. neurotensin. Members of the EGF family have similar structures and can bind to EGFR, members of the IGF family can bind IGF receptor (IGFR), and neurotensin can bind neurotensin receptor-1 (NTSR1). Possible points for ligand attachment are for example, but not limited to, N-terminus of any Sm/LSm protein, C-terminus of any Sm/LSm protein, and within the loop between β strands 3 and 4 of any Sm/LSm protein. The loop between β strands 3 and 4 can be modified to optimize for RNA protection from degradation by nucleases. Insertion of domains or long loops occurred in nature at these three positions without affecting their ability to form rings with other Sm/LSm proteins (e.g. LSm11 of U7 snRNP has a domain at the N-terminus of the Sm core and a long loop insert between β strands 3 and 4). In one embodiment of the present invention, EGF is attached onto the C-terminus of SmD2 protein to form SmD2-EGF (SEQ ID NO: 8), and the Sm binding sequence is attached at the 5′-end of a short-hairpin RNA (shRNA). The shRNA delivery agent is named as “short-hairpin ribonucleoprotein complex” (shRNP) (FIG. 2B). As RNA containing the Sm binding sequence will bind to the Sm proteins, attaching this short stretch of RNA sequence to various forms of RNA molecules (e.g. siRNA, saRNA, mRNA) should form a ribonucleoprotein complex with Sm proteins in vitro for delivery into cells or in vivo. Hence, such various forms of RNA molecules are further embodiments of the present invention. In several embodiments of the present invention, the siRNP and shRNP are successfully reconstituted and siRNA and/or shRNA are delivered into cells for gene silencing. This shRNA or siRNA delivery method does not utilize a DNA vector that is usually delivered using lipid-based carrier for expression of siRNA or shRNA in the nucleus. This invention allows for the use of recombinant and modified Sm proteins as agents to directly deliver siRNA or shRNA for eliciting RNAi-based gene silencing in the cytoplasm. It is also envisioned that the in vitro reconstituted ribonucleoprotein complexes be delivered in vivo through, but not limited to, peritumoral, intravenous, or subcutaneous injection for disease treatment.

### Examples

The following examples are intended to aid the understanding and enablement of certain embodiments of the present invention, which should not be intended to limit the scope of the present invention.

In Vitro Assembly of shRNP

As proof-of-principle that the Sm proteins can be engineered as RNA delivery agent, the Sm proteins were engineered from the U4 snRNP to deliver an shRNA that targets KRAS gene for knockdown via RNA interference. Separately, recombinant proteins were co-expressed and purified for the subcomplexes SmD1/SmD2-EGF complex, SmD3/SmB₁₋₉₅ complex, and SmG/SmE complex. The C-terminus of SmD2 was fused with an epidermal growth factor (EGF) as ligand for EGF receptor (EGFR) for cellular uptake via endocytosis. To improve solubility of the SmD1/SmD2-EGF complex, a solubility tag SUMO was also fused onto the N-terminus of SmD1 for expression and the tag was later removed by addition of ULP1 protease during assembly. In order for the shRNA targeting KRAS to bind to the Sm proteins, a Sm binding sequence was added at the 5′-end of the shRNA in the present design. Additionally, a 6-FAM fluorescent label was added at the 5′-end of the shRNA for tracking of the shRNA in cells.

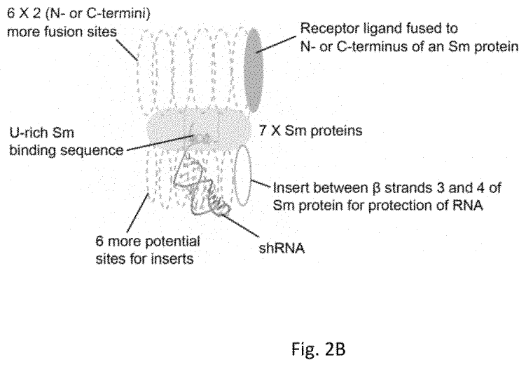

The Sm protein subcomplexes were mixed with the shRNA and the shRNP complex purified by size exclusion chromatography (FIG. 3). The purified complex had a 260/280 nm absorbance ratio of 1.5 (>1), indicating the presence of RNA. In this version of the shRNP complex, a 6-Sm protein-shRNA complex was obtained even though normally U4 snRNP contains 7 Sm proteins (SmF is not included). The presence of the six Sm proteins was confirmed by SDS-PAGE analysis (FIG. 2).

### Cellular Uptake

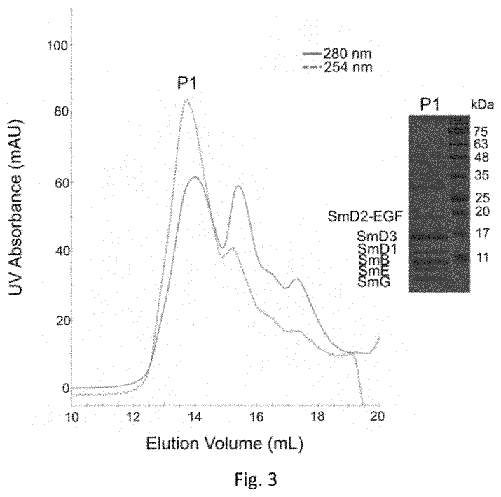

To investigate if shRNP can be uptaken into cells, it was tested on A549 lung adenocarcinoma cells. A549 cells are K-Ras positive. The cells were incubated in media containing shRNP at 123 nM final concentration. As control, the cells were incubated in media with the corresponding amounts of the Sm proteins used to assemble the shRNP complex. After two days, the media were replaced with fresh media and confocal fluorescence microscopy was performed. Under confocal microscopy, it was observed that cells incubated with shRNP contained diffused FAM fluorescence signals in the cytoplasm, suggesting that the FAM-tagged shRNA had entered the cell cytoplasm (FIG. 4). In the Sm protein only control, only low amounts of background fluorescence signals were observed.

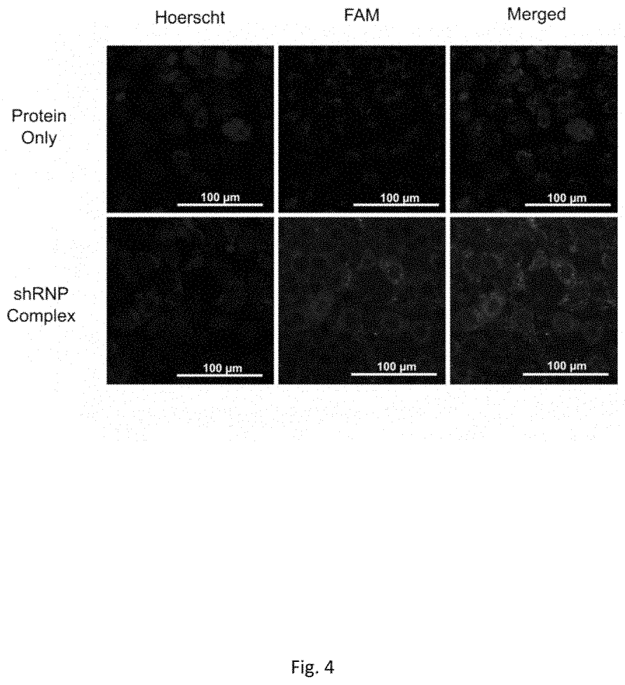

To further confirm that the observation for the protein only control was just background noise, the tests were repeated with shRNA only (without complexation with Sm protein), protein only, and buffer controls. Cells from all these controls also gave similar background speckled fluorescence when using FAM channel that did not diffuse over the cell cytoplasm (FIG. 5), suggesting that the diffused fluorescence signals in the cell cytoplasm of cells incubated with shRNP were derived from the FAM-tagged shRNA that had entered cells (FIG. 4). From these experiments, it is concluded that the shRNP complex is able to deliver shRNA into the cells.

### KRAS Gene Expression Analysis

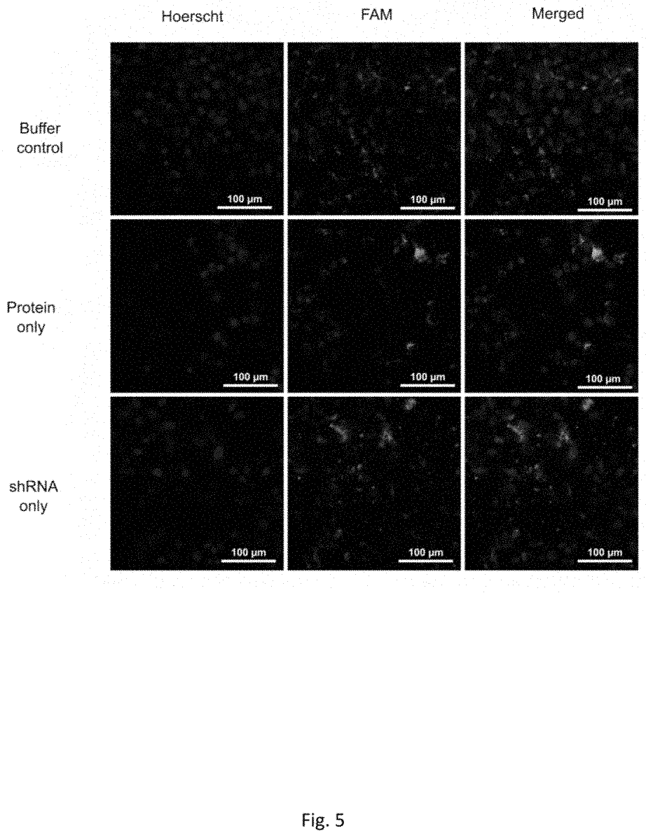

To investigate if the shRNA targeting KRAS that entered A549 cells in the form of shRNP can silence KRAS via RNA interference, quantitative polymerase chain reaction (qPCR) was performed to check for KRAS gene expression. A549 cells were first incubated for 4 days with 123 nM shRNP, corresponding amounts of Sm proteins, and buffer control, then qPCR was performed on KRAS RNA using two different sets of primer pairs. Results from our qPCR studies showed that KRAS RNA levels, relative to buffer control, decreased for both cells incubated with shRNP and Sm proteins (FIGS. 6A & 6B). The percentages of change in KRAS expression for shRNP incubated cells were 84% (primer pair 1 (SEQ ID NOs: 29 and 30)) (FIG. 6A) and 74% (primer pair 2) (SEQ ID NOs: 31 and 32) (FIG. 6B) of buffer control. However, the results showed even more reduction in KRAS gene expression for the Sm protein only sample when compared to buffer control (55% expression for primer pair 1 and 50% for primer pair 2 (SEQ ID NOs: 31 and 32)) (FIGS. 6A & 6B). It was observed that Sm proteins without shRNA reduced KRAS expression but there was no additive effect when shRNA was present. It is only speculative that there is interference between the ligand EGF and KRAS expression because K-Ras (product of the gene KRAS) and EGFR are on the same cell signaling pathway. In order to study the RNAi effect of shRNP without such interference, another target that is not directly affected by the ligand and cell receptor binding and activation will be needed. These results also provided insight into the optimization of shRNP design, which is to avoid using a ligand that could potentially interfere with the silencing target.

### Cell Viability

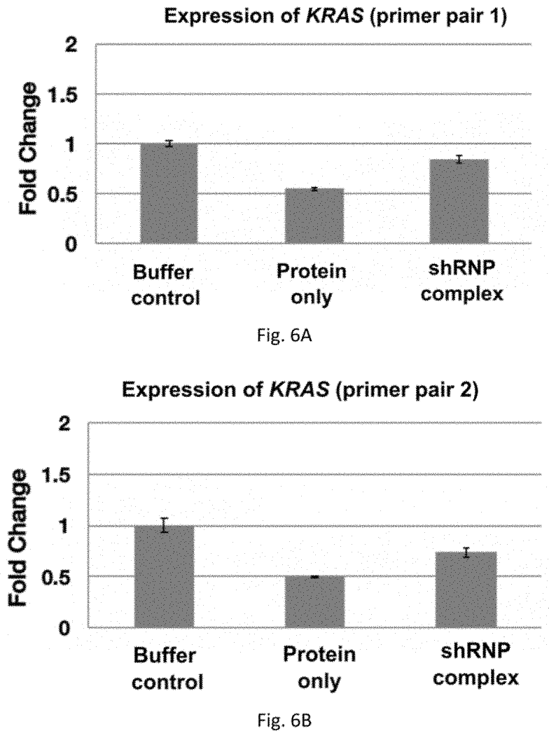

To investigate the effects of shRNP on A549 cell viability, MTT assays were performed on A549 cells incubated with 123 nM and 246 nM shRNP, corresponding Sm proteins only (without shRNA), and buffer control. After 4 days of incubation, MTT assays were performed. Compared to buffer control (100%), cell viability for cells incubated with 123 nM shRNP was 93.33% while protein only control was 108.55% (FIG. 7). For cells incubated with 246 nM shRNP, its viability compared to buffer only control was only 23.53%. However, the corresponding cells incubated with protein control also had significant reduction in cell viability, 34.56%, suggesting that at higher concentration, the protein carrier could be toxic to A549 cells. These results suggest that at lower concentration of shRNP, in which protein carrier is not toxic, there was only a mild negative effect on A549 lung cancer cell viability. It was previously reported that shRNA knockdown of KRAS in A549 cells only had no significant cell viability reduction (Singh et al., 2009, A Gene Expression Signature Associated with “K-Ras Addiction” Reveals Regulators of EMT and Tumor Cell Survival. Cancer Cell), supporting the observations described herein.

egfp as Target for Silencing

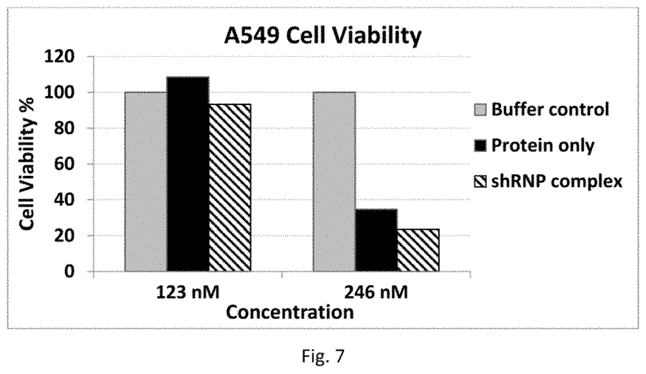

Due to interference between EGFR activation by EGF ligand and KRAS gene expression, it could not be conclusively shown that shRNP has RNAi effect. In order to show that the shRNA delivered by shRNP into cells can lead to gene silencing, shRNP carrying a shRNA targeting egfp was assembled. As the product of egfp gene is enhanced green fluorescence protein (eGFP) that produces green fluorescence, eGFP can serve as a reporter; green fluorescence reduction is expected if egfp gene silencing occurs. In this assembly, the same set of Sm proteins and modifications, including the EGF ligand fusion at the C-terminus of SmD2, were used. The shRNA containing a fragment of egfp mRNA sequence was designed and attached with an Sm binding sequence on its 5′-end. The fluorescence tag was changed to DY547 (red) so that it will not interfere with the fluorescence signal from eGFP (green). Using similar methods as described above, the shRNP complex targeting egfp was assembled and verified by size exclusion chromatography, 260/280 ratio (1.5) and SDS-PAGE analysis (FIG. 8).

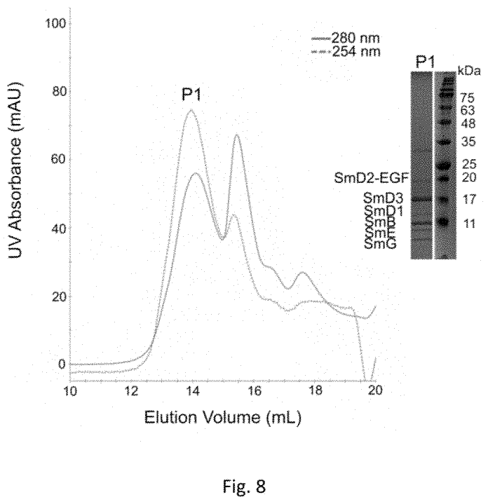

100 nM of shRNP targeting egfp was incubated with 293T-eGFP cells that express endogenous eGFP. Cells were set up for incubation with 100 nM each of Sm protein subcomplexes without shRNA (protein only), 100 nM shRNA only and Buffer Control. After two days of incubation, the media were replaced with fresh media, and confocal microscopy studies were performed (FIG. 9). In the shRNP complex sample, DY547 channel fluorescence signals were observed in the cytoplasm of the majority of cells but the DY547 signals were absent in the Protein only and Buffer Control. In the controls, majority of the cells had eGFP fluorescence signals. Remarkably, it was observed that the number of cells with eGFP signals markedly reduced in the sample incubated with shRNP complex when compared with Buffer Control and Protein only controls. It was also observed in the shRNP complex sample that cells that showed DY547 fluorescence signals had unobservable or reduced eGFP fluorescence signals, and cells that did not have DY547 signals showed high intensity of eGFP signals. These observations suggest that the shRNP complex targeting egfp, when delivered into cells, can lead to reduction of eGFP levels in the cells. In the shRNA only sample, however, DY547 signals were also present in some cells but there was no significant reduction in eGFP signals these cells. This observation suggests that the egfp expression was not silenced in the shRNA only sample. The observation of DY547 signals in cells in the shRNA only sample could be attributed to the presence of DY547 fluorescent dye molecules that are detached from degraded RNA. Cy3 dye from Cy3-tagged RNAs could enter cells when the RNA is degraded by nucleases. DY547 is a Cy3 alternative and is structurally similar to Cy3. DY547 and Cy3 possess a positive charge at neutral pH, which aids their entry into cells. Based on the lack of reduction of eGFP signals in cells stained with DY547 in the shRNA only sample, it is inferred that the DY547 signals came from DY547 dye as a result of RNA degradation; the RNA most likely did not enter cells thus no reduction in eGFP fluorescence. It reaches a conclusion that shRNA, only when delivered by shRNP, can have a gene silencing effect.

In the previous embodiments of the present invention, the inventors showed that shRNP containing 6 Sm proteins (SmD1, SmD2-EGF, SmD3, SmB₁₋₉₅, SmG and SmE) and an shRNA either targeting KRAS or egfp can be uptaken by cells.

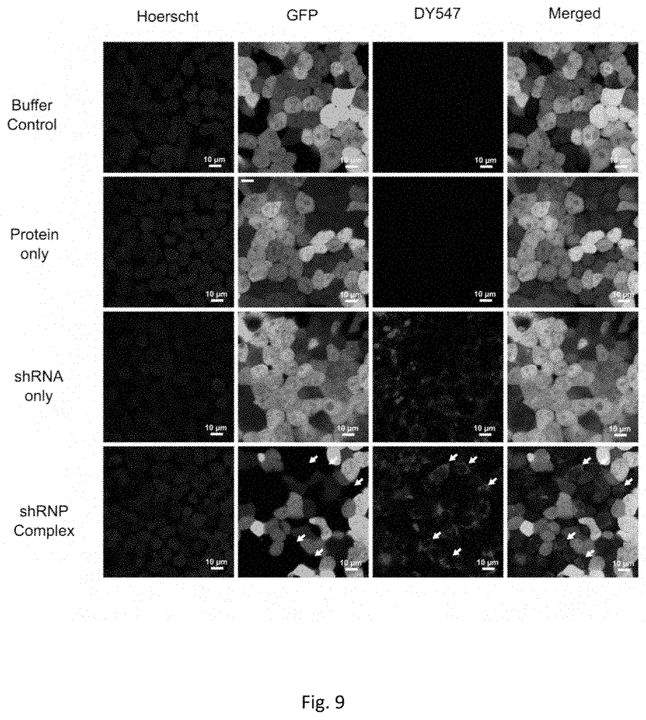

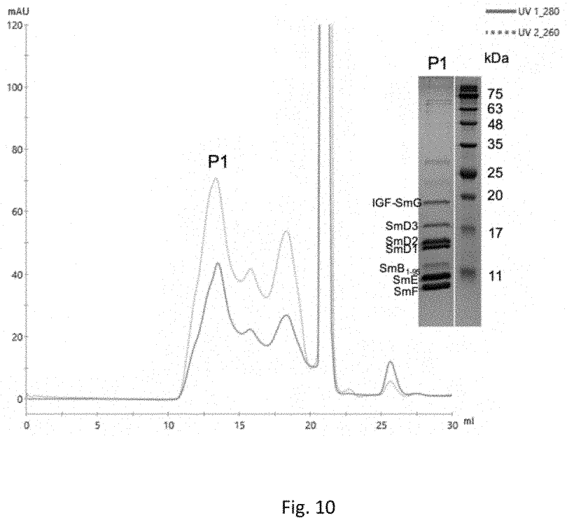

In this embodiment of the present invention, the inventors show that an shRNP consisting of (1) 7 Sm proteins, (2) a different ligand for receptor-mediated endocytosis, (3) and the receptor ligand being on a different Sm protein can also be reconstituted and elicit a gene knockdown effect. The inventors performed in vitro reconstitution of another shRNP complex consisting of SmD1, SmD2, SmD3, SmB₁₋₉₅ (a variant of SmB (SEQ ID NO: 3), IGF-SmG (SEQ ID NO: 39) (SmG with its N-terminus fused with insulin-like growth factor 1, IGF1, residues 49-118 (SEQ ID NO: 40)), SmE, and SmF and an shRNA targeting KRAS labelled with FAM on the 5′-end (SEQ ID NO: 36) (This shRNA also contains a 2-nucleotide overhang on the 3′-end that does not form base-pairing) (FIG. 10). The inventors also reconstituted another version of RNA delivery agent containing the above proteins (SmD1, SmD2, SmD3, SmB₁₋₉₅, IGF-SmG, SmE, and SmF) and a small-interfering RNA (siRNA) targeting KRAS (SEQ ID NOS: 37 and 38) (FIG. 11). siRNA consists of two strands of short RNAs with the Sm-binding site on the 5′-end of the intended guide strand. The guide strand also contains a 2-nucleotide overhang that does not form base pairing on the 3′-end.

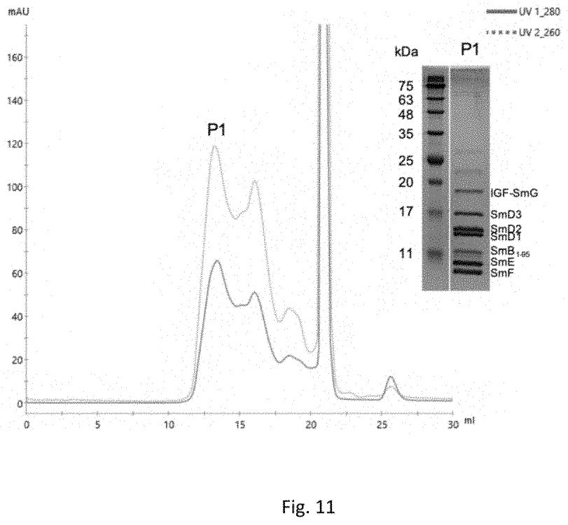

The inventors treated A549 cells that are K-Ras positive with 100 nM shRNP, siRNP, shRNA only, siRNA only, protein carrier only, or buffer control and incubated the cells for 48 hours before performing quantitative PCR to investigate KRAS gene expression (FIG. 12). qPCR results showed that the shRNP had 61% KRAS expression relative to buffer control but there is no significant difference from shRNA control (65%). KRAS expression of cells treated with siRNP had 66% expression relative to buffer control, but not significantly lower than siRNA control (60%). The protein carrier control showed KRAS expression of 83% relative to buffer control. Overall, these results showed that while samples containing shRNA or siRNA with or without being complexed by the Sm protein carrier gave lower KRAS gene expression, the knockdown effect of the Sm protein as carrier for shRNA or siRNA delivery could not be demonstrated. However, the inventors were able to demonstrate that a 7-protein complex with shRNA or siRNA could be reconstituted.

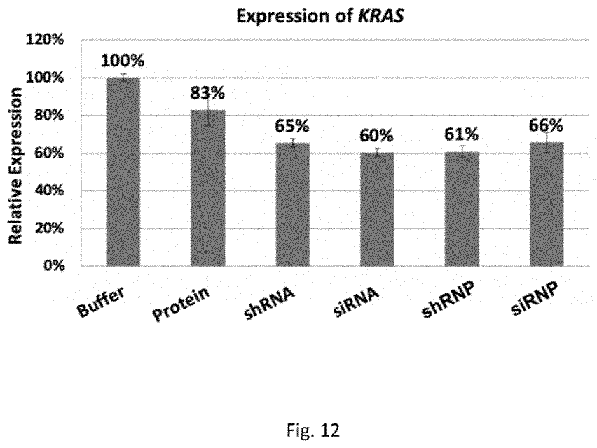

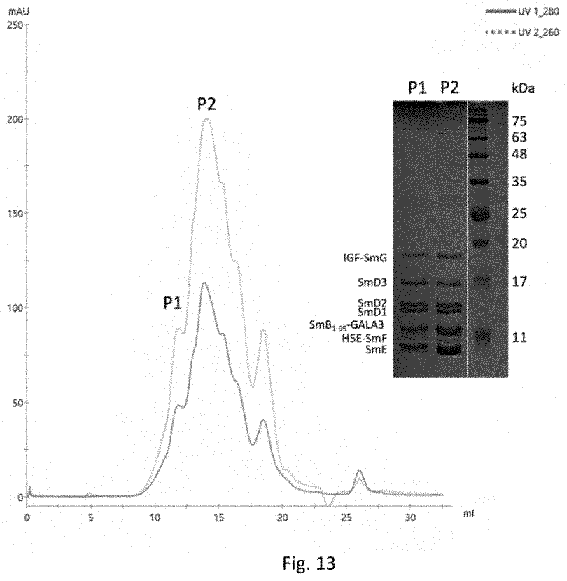

Because the inventors previously were able to observe cellular uptake of shRNPs by confocal microscopy but their gene silencing effects were not definitively demonstrated by qPCR, the present disclosure proposes that the uptaken shRNPs can be trapped in endosomes. Endosomal escape is a known hurdle in drug delivery. Endosomal escape peptides (EEP) have been reported to be able to disrupt endosomes allowing drugs to be released into the cytoplasm. To investigate if fusing EEPs to shRNP proteins will allow RNA to break free from endosomes and elicit stronger RNAi effects, the inventors fused two different EEPs onto the two different Sm proteins: GALA3 (SEQ ID NO: 44) onto the C-terminus of SmB₁₋₉₅ (SmB₁₋₉₅-GALA3 (SEQ ID NO: 41)) and H5E (SEQ ID NO: 43) onto the N-terminus of SmF (H5E-SmF (SEQ ID NO: 42)). The inventors reconstituted two versions of shRNPs: (1) shRNP with SmB₁₋₉₅-GALA3 as well as H5E-SmF (SmD1, SmD2, SmD3, SmB₁₋₉₅-GALA3, IGF-SmG, SmE, and H5E-SmF, FAM-shRNA_{KRAS}) (FIG. 13); and (2) shRNP with H5E-SmF (SmD1, SmD2, SmD3, SmB_{1-95,} IGF- SmG, SmE, and H5E-SmF, FAM-shRNA_{KRAS}) (FIG. 14). When purifying the shRNPs containing EEPs by size exclusion chromatography, the inventors observed two major peaks in their size exclusion chromatography profiles. The inventors collected and concentrated the fractions corresponding to the two peaks separately. SDS-PAGE analysis of shRNP with both SmB₁₋₉₅-GALA3 and H5E-SmF showed that the first peak, which corresponds to a higher molecular weight complex, was missing H5E-SmF while the second peak contains all 7 proteins, i.e. including both SmB₁₋₉₅-GALA3 and H5E-SmF. For the second shRNP version, which contains just one type of EEP, i.e. H5E on SmF, both peaks contain 7 proteins (SmD1, SmD2, SmD3, SmB₁₋₉₅, IGF-SmG, SmE, and H5E-SmF).

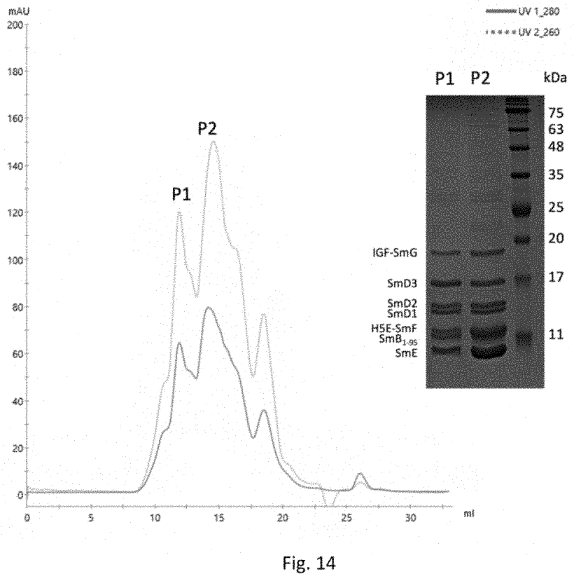

The inventors then treated the A549 cells with 100 nM shRNPs from different peaks along with buffer, protein, and shRNA controls. The inventors performed qPCR on these samples to investigate their gene knockdown effects (FIG. 15). qPCR results showed that the sample from peak 2 from size exclusion chromatography of shRNP with both GALA3 and H5E could reduce KRAS expression levels to just 45% of buffer control and also significantly lower than shRNA and protein controls (80% and 84% respectively). However, the sample treated with peak 1 from size exclusion chromatography of shRNP with GALA3 and H5E (though H5E was not detected on SDS-PAGE) did not show significant difference in KRAS expression levels (85%) when compared with RNA and protein controls (80% and 84% respectively). For shRNP containing only H5E (without GALA3), both peaks 1 and 2 from size exclusion chromatography could reduce gene expression of KRAS to 51 and 42% respectively relative to buffer control, also significantly lower than shRNA and protein control (80% and 91% respectively). These results indicate that adding an endosomal escape peptide such as H5E to shRNP can improve its gene knockdown effect.

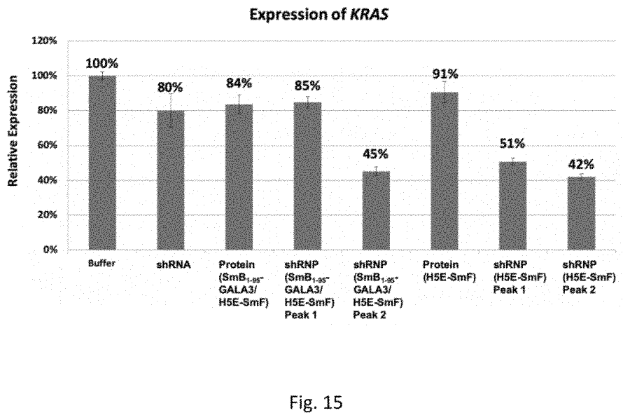

To confirm cellular uptake of shRNPs containing EEPs with IGF as receptor ligand, the inventors performed confocal microscopy studies on cells incubated with 100 nM shRNP containing both GALA3 and H5E (Peak 2) for 48 hours. The confocal microscopy images showed that cells incubated with shRNP containing GALA3, H5E and IGF had a stronger diffused fluorescence staining corresponding to FAM relative to buffer, protein and shRNA controls, which had weaker background fluorescence signals, suggesting that the shRNP complex aided cellular uptake of shRNA (FIG. 16). These results showed that switching the ligand to IGF on a 7-protein shRNP complex can be uptaken by cells.

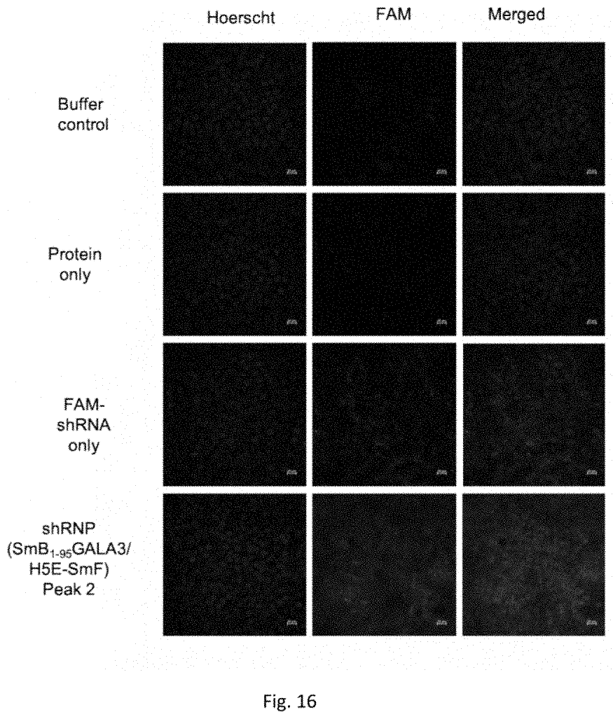

To show that the ribonucleoprotein core can also carry an siRNA for gene silencing, the inventor reconstituted a siRNP-IGF-H5E consisting of SmD1, SmD2, SmD3, SmB₁₋₉₅, IGF-SmG, SmE, and H5E-SmF) and a small-interfering RNA (siRNA) targeting KRAS (SEQ ID NOS: 37 and 38). The inventor then treated A549 cells with 100 nM siRNP-IGF-H5E along with shRNP-IGF-H5E, buffer, protein, siRNA, and shRNA only controls for 48 hours. The inventors performed qPCR on these samples to investigate their gene knockdown effects (FIG. 17). qPCR results showed that siRNP-H5E reduced KRAS expression levels to 65% relative to buffer control, which is comparable to shRNP (56% relative to buffer control). Both siRNP and shRNP showed reduction in KRAS expression to levels that are lower than protein, siRNA, and shRNA controls (113%, 87% and 97% relative to buffer control, respectively). Therefore, the inventors concluded that siRNP containing an EEP, like an shRNP containing an EEP, is also able to deliver siRNA for gene silencing.

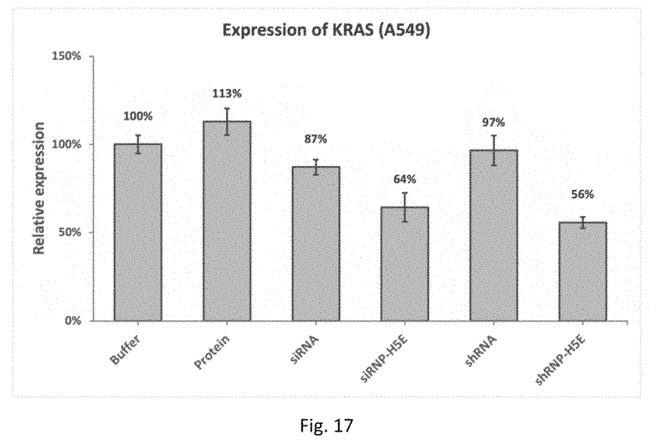

To investigate whether using different ligands could result in different levels of internalization of the shRNPs into cells, the inventors used flow cytometry to quantify the levels of fluorescent-tagged shRNA in cells delivered by different shRNPs. The inventors assembled three versions of shRNPs: (1) shRNP-IGF-H5E consisting of SmD1, SmD2, SmD3, SmB₁₋₉₅, IGF-SmG, SmE, and H5E-SmF and a DY547-tagged shRNA targeting KRAS (SEQ ID NO: 27); (2) shRNP-IGF/EGF-H5E consisting of IGF-SmD1, SmD2, SmD3, SmB₁₋₉₅-H5E, IGF-SmG, SmE-EGF, and SmF and a DY547-tagged shRNA targeting KRAS (SEQ ID NO: 27); (3) shRNP-EGF-H5E consisting of SmD1, SmD2, SmD3, SmB₁₋₉₅-H5E, SmG, SmE-EGF, and SmF and an DY547-tagged shRNA targeting KRAS (SEQ ID NO: 27). The inventors treated A549 cells with 100 nM of each shRNP and performed flow cytometry to quantify the fluorescence intensity of each sample at time points 1 h, 2 h, and 4 h post-treatment with shRNP (FIG. 18). Overall, cells incubated with shRNP complexes have higher fluorescence intensity (202531-923000) relative to buffer control (1 h: 1428; 2 h: 1350; 4 h: 1405) and shRNA only control (1 h: 4435; 2 h: 11380; 4 h: 21168). Cells incubated with shRNP-IGF-H5E complex has higher fluorescence intensity (1 h: 612000; 2 h: 916000; 4 h: 542000) compared to shRNP-EGF-H5E (1 h: 202531; 2 h: 310042; 4 h: 299605), indicating that using IGF as ligand causes a higher cellular uptake of shRNP than using EGF as a ligand. Cells incubated with the shRNP-IGF/EGF-H5E has similar fluorescence intensities (1 h: 530000; 2 h: 923000; 4 h: 506000) as cells incubated with shRNP-IGF-H5E, suggesting that there is no synergistic increase in cellular uptake with the simultaneous presence of IGF and EGF ligands. These results also further support that the Sm protein carriers function as delivery agents.

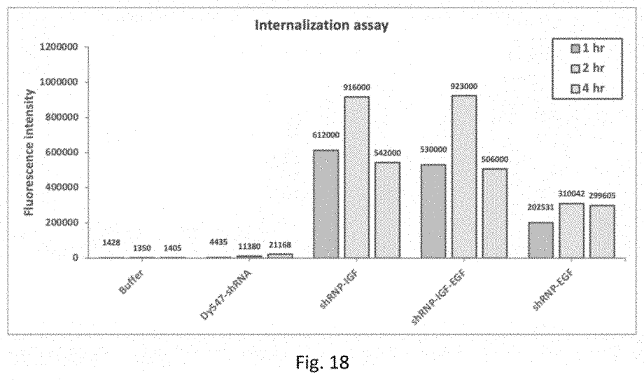

To investigate whether silencing KRAS will have an effect on cancer cell viability, the inventors investigated KRAS silencing and cell viability effects of siRNPs on a human colorectal carcinoma cell line HCT116. HCT116 is K-Ras-dependent for progression. The inventors assembled three versions of siRNPs: (1) siRNP-IGF-H5E consisting of SmD1, SmD2-IGF, SmD3, SmB-H5E, SmG, SmE, and SmF, and a small-interfering RNA (siRNA) targeting KRAS (SEQ ID NOs: 37 and 38); (2) siRNP-EGF-H5E consisting of SmD1, SmD2, SmD3, SmB₁₋₉₅-H5E, SmG, SmE-EGF, and SmF and a small-interfering RNA (siRNA) targeting KRAS (SEQ ID NOs: 37 and 38); and (3) siRN4P-IGF-H5E, a ribonucleoprotein complex consisting of 4 Sm proteins instead of 7 (SmD1, SmD2-IGF, SmD3, SmB-H5E and SmE). The inventors treated HCT116 cells with 100 nM of each of the siRNP, along with siRNA, corresponding proteins, and buffer only controls for 48 hours. After that, the inventors performed qPCR to investigate KRAS expression levels (FIG. 19). Relative to buffer control, all three types of siRNPs reduced KRAS expression levels (siRNP-IGF-H5E, 58%; siRNP-EGF-H5E, 51%; and siRN4P-IGF-H5E 66%) relative to the buffer control. The siRNA and protein only controls showed higher expression of KRAS than the samples treated with siRNPs (siRNA, 73%; 7-protein with IGF, 76%; 4-protein with IGF, 74%; and 7-protein with EGF, 89%). Based on these observations, it was concluded that siRNPs containing EGF or IGF can lead to gene silencing. The inventors also showed that a ribonucleoprotein complex consisting of four Sm proteins can also reduce gene expression.

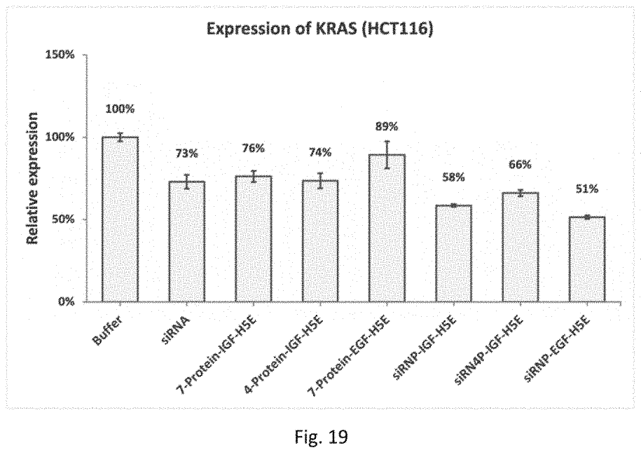

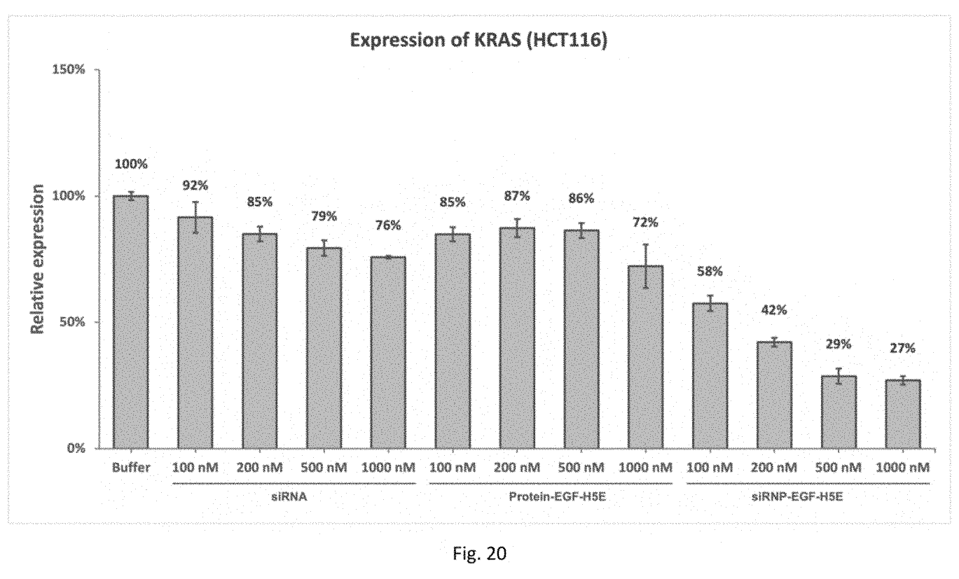

To investigate the dose-dependent effects of siRNP on KRAS gene expression and HCT116 cell viability, the inventors treated HCT116 with different concentrations of siRNP-EGF-H5E (100 nM, 200 nM, 500 nM and 1000 nM), along with siRNA and protein only controls with corresponding concentrations for 48 hours. After that, the inventors performed qPCR to quantify KRAS gene expression (FIG. 20). The inventors observed that increasing concentrations of siRNP-EGF-H5E resulted in decreasing KRAS gene expression (58%, 42%, 29% and 27% respectively). The siRNA and protein only controls showed KRAS levels between 72-92%. These results demonstrated that siRNP-EGF-H5E reduced KRAS gene expression in a dose-dependent manner. To show that silencing of KRAS by siRNP-EGF-H5E can reduce cancer cell progression, the inventors treated HCT116 with different concentrations of siRNP-EGF-H5E (100 nM, 200 nM, 500 nM and 1000 nM), along with the corresponding siRNA and protein only controls, for 72 hours and determined the cell viability by MTT assay (FIG. 21). The inventors observed a dose-dependent trend of decreasing cell viability with increasing concentration of siRNP-EGF-H5E (85%, 79%, 61% and 38% relative to buffer control (100%), respectively). With the exception of the sample treated with protein only at 1000 nM concentration, which has a relative KRAS expression of 73%, all other siRNA and protein only controls have higher KRAS levels than siRNP-EGF-H5E, between 90-131%. These observations indicated that the siRNP-EGF-H5E can reduce cancer cell progression of a K-Ras-dependent cancer cell line HCT116, thus showing potential as an anti-cancer therapeutic.

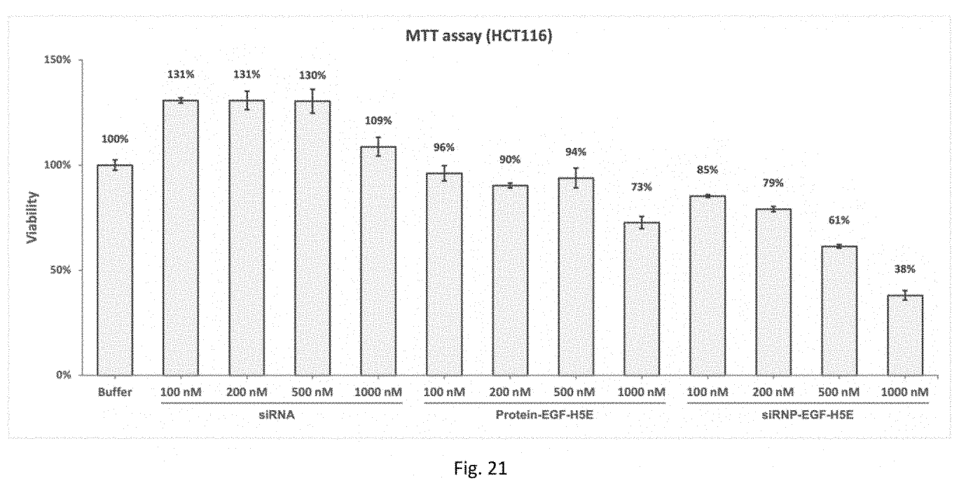

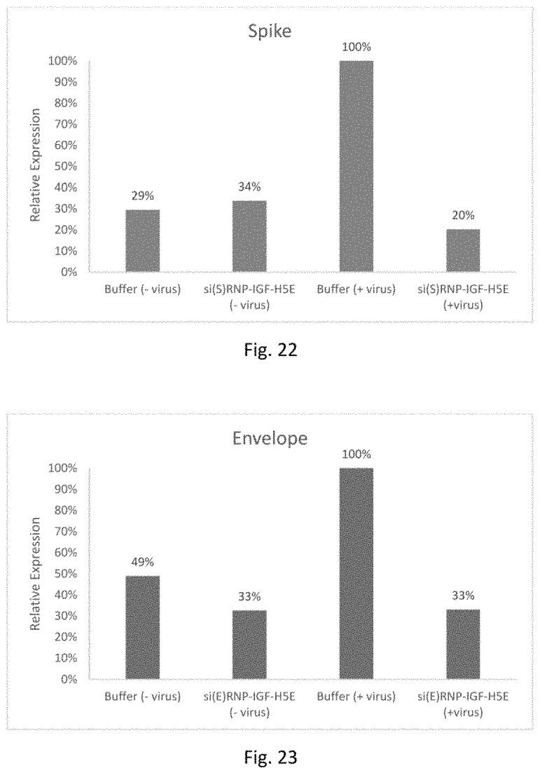

To demonstrate that the invention can also be used as potential antiviral therapeutics, the inventors assembled siRNPs targeting Human Coronavirus 229E (HCoV-229E) Spike (5) and Envelope (E) genes. The S gene encodes for the coronavirus Spike protein while the E gene encodes for the coronavirus Envelope protein. To target the HCoV-229E S gene, the inventors assembled si(S)RNP-IGF-H5E consisting of SmD1, SmD2, SmD3, SmB₁₋₉₅, IGF-SmG, SmE, and H5E-SmF, and a small-interfering RNA (siRNA) targeting the Spike (5) gene (SEQ ID Nos: 45 and 46 for si(S)-(+) and si(S)-(−)). To target the HCoV-229E E gene, the inventors assembled si(E)RNP-IGF-H5E consisting of SmD1, SmD2, SmD3, SmB₁₋₉₅, IGF-SmG, SmE, and H5E-SmF, and a small-interfering RNA (siRNA) targeting E (SEQ ID Nos: 47 and 48 for siE-(+) and siE-(−)). To investigate whether the siRNPs can silence viral genes in cells subjected to viral infection, the inventors first treated VeroE6 Cells with HCoV-229E by incubating the cells with the virus for 2 hours, after which the inventors removed the virus-containing media and replenished fresh media containing 500 nM of each siRNP (si(S)RNP-IGF-H5E and si(E)RNP-IGF-H5E), along with buffer controls. The inventors also set up controls with VeroE6 cells without being exposed to HCoV-229E but treated with 500 nM of each siRNP (si(S)RNP-IGF-H5E and si(E)RNP-IGF-H5E) or buffer. After 48 hours of treatment with siRNPs or corresponding controls, the RNA from each sample was extracted for qPCR analysis (FIG. 22). The qPCR analyses showed that the si(S)RNP-IGF-H5E complex reduced the expression of the S gene in the sample that was exposed to HCoV-229E to 20% relative to that of the infected buffer control (100%). The S gene expression levels are comparable to the levels for samples that have not been exposed to HCoV-229E (buffer: 29%; si(S)RNP-IGF-H5E: 34%), indicating that the si(S)RNP-IGF-H5E reduced the relative expression of the S gene to the levels equivalent to uninfected samples. Similar trends were observed for the expression levels of the E gene (FIG. 23). The qPCR analyses showed that the si(E)RNP-IGF-H5E complex reduced the levels of the E gene expression in the infected sample to 33% relative to that of the infected buffer control (100%). The S gene expression levels are also comparable to the levels of the uninfected samples (buffer: 49%; si(S)RNP-IGF-H5E: 33%), indicating that the si(E)RNP-IGF-H5E reduced the relative expression of the E gene to the levels equivalent to the uninfected samples. In conclusion, the inventors demonstrated that the siRNPs can silence viral gene expression in infected cells and thus can potentially be developed into an antiviral therapy.

Overall, the inventors demonstrated that their modular and customizable RNAi delivery agent can be modified to enhance its gene knockdown efficacies. With these results, this embodiment of the present invention demonstrated that 1) a 7-Sm or 4-Sm protein shRNP or siRNP complex can be reconstituted for RNA delivery, 2) the receptor ligand can be moved to another Sm protein and still allows for siRNP and shRNP to deliver RNA and knockdown genes, 3) cellular uptake can still occur using a different receptor ligand, 4) adding an EEP(s) to shRNP can enhance its gene silencing effects, 5) an oncogene silenced by the siRNP can reduce cancer cell viability, and 6) viral genes can be silenced in infected cells using the siRNPs.

### Materials and Methods

### Expression and Purification of SmD1/SmD2/SmD2-EGF

Construct encoding for SmD1 (synthesized by Genscript) was cloned into pET28a-sumo vector encoding for an N-terminal hexahistidine-tag followed by a SUMO-tag. Construct encoding for SmD2 or SmD2-epidermal growth factor (SmD2-EGF) (synthesized by Genscript) was cloned into MCS2 of pCDFDuet-1. Both plasmids were then co-transformed in E. coli BL21(DE3) and the cells were grown in 2×YT media at 37° C. with 50 μg/ml kanamycin, 100 μg/ml streptomycin, and until OD600 reached 0.6-0.8. The cells were then induced using a final concentration of 0.5 mM isopropyl β-d-1-thiogalactopyranoside (IPTG) and were grown at 20° C. for 18 h. The cells were harvested by centrifugation. The cell pellets were resuspended in lysis buffer (20 mM Tris, pH7.5, 1 M NaCl, 10 mM imidazole, 5% (v/v) glycerol, 10 mM 3-mercaptoethanol (BME) and 17.8 μg/mL phenylmethylsulfonyl fluoride (PMSF)) and lysed by sonication. Cell lysate was cleared by centrifugation and loaded onto a HisTrap HP column (Cytiva). The column was then washed with 20 column volumes (CV) of wash buffer (20 mM Tris, pH 7.5, 1 M NaCl, 40 mM imidazole) and eluted with elution buffer (20 mM Tris, pH 7.5, 1 M NaCl, 500 mM imidazole). The eluted protein was diluted 3 times with buffer A (20 mM HEPES, pH 7.5, 5 mM DTT) and further purified using a 5 ml HiTrap Heparin column (GE Healthcare) equilibrated with 85% buffer A and 15% buffer B (20 mM HEPES, pH 7.5, 2 M NaCl, 5 mM DTT). The protein was eluted using a gradient from 15% to 100% buffer B. The SUMO-tag on SmD1 was then removed by incubating with ULP1 protease overnight at 4° C. The untagged SmD1/SmD2 complex was subsequently purified by reverse His-tag purification using 1 ml HisTrap HP column (Cytiva). Fractions of interest were concentrated and store at −80° C.

Expression and Purification of His-SmD3/SmB₁₋₉₅-GALA3, His-SmD3/SmB₁₋₉₅-H5E and His-SmD3/SmB₁₋₉₅

The construct for N-terminal hexahistidine tagged SmD3 was cloned into pET28a and the construct encoding for SmB residues 1-95 (SmB₁₋₉₅), SmB₁₋₉₅-GALA3 fusion or His-SmD3/SmB₁₋₉₅-H5E protein was cloned into MCS2 of pCDFDuet-1. Both plasmids were then co-transformed into E. coli BL21 (DE3) cells and grown in 2×YT media supplemented by 50 μg/ml kanamycin and 100 μg/ml streptomycin at 37° C., expression was induced with a final concentration of 0.5 mM IPTG when its OD₆₀₀ reached 0.6-0.8. The cells were grown at 20° C. for 18 hours. Cells were harvested by centrifugation and the cell pellets were resuspended in lysis buffer (20 mM Tris, pH 7.5, 500 mM NaCl, 10 mM imidazole, 5% (v/v) glycerol, 10 mM BME and 17.8 μg/mL PMSF) and lysed by sonication. The cell lysate was cleared by centrifugation and loaded onto a 5 ml HisTrap HP column (GE Healthcare). The column was then washed with 20 CV of wash buffer (20 mM Tris, pH 7.5, 500 mM NaCl, 40 mM imidazole) and eluted with elution buffer (20 mM Tris, pH 7.5, 500 mM NaCl, 500 mM imidazole). The eluted protein was further purified by using a 5m1 HiTrap Heparin column (GE Healthcare) equilibrated with 60% buffer A (20 mM HEPES, pH 7.5 and 5 mM DTT) and 40% buffer B (20 mM HEPES, pH 7.5, 1 M NaCl, 5 mM DTT). The protein was eluted with a gradient from 40% to 100% buffer B. Fractions of interest were concentrated and stored at −80° C.

### Expression and Purification of SmG/SmE

The construct for C-terminal hexahistidine-tagged SmG was cloned into pET26b and SmE was cloned into MCS1 of pCDFDuet-1 vector. Both plasmids were then co-transformed into E. coli Rosetta (DE3) pLysS cells and grown in 2×YT media supplemented by 50 μg/ml kanamycin, 100 μg/ml streptomycin and 34 μg/ml chloramphenicol at 37° C. When the OD₆₀₀ the culture reached 0.6-0.8, the cells were induced by adding a final concentration of 0.5 mM IPTG and grown at 20° C. for 18 h. The cells were harvested, and the complex was purified as described for the His-SmD3/SmB 1-95 complex.

### Expression and Purification of IGF-SmG-His/SmE/H5E-SmF, IGF-SmG-His/SmE/SmF and SmG-His/SmE-EGF/SmF

The construct for C-terminal hexahistidine-tagged SmG, or hexahistidine-tagged SmG with insulin-like growth factor 1 residues 49-118 (IGF) fused to its N-terminus, was cloned into pET26b, while SmE or SmE-EGF and SmF or H5E-SmF was cloned into MCS1 and MCS2 of pCDFDuet-1 respectively. Both plasmids were then co-transformed into E. coli BL21 cells and grown in 2×YT media supplemented by 50 μg/ml kanamycin and 100 μg/ml streptomycin at 37° C., expression was induced with a final concentration of 0.5 mM IPTG when its OD₆₀₀ reached 0.6-0.8. The cells were grown at 20° C. for 18 hours. Cells were harvested by centrifugation and the cell pellets were resuspended in lysis buffer (20 mM Tris, pH 7.5, 500 mM NaCl, 10 mM imidazole, 5% (v/v) glycerol, 10 mM BME and 17.8 μg/mL PMSF) and lysed by sonication. The cell lysate was cleared by centrifugation and loaded onto a 5 ml HisTrap HP column (Cytiva). The column was then washed with 20 CV of wash buffer (20 mM Tris, pH 7.5, 500 mM NaCl, 40 mM imidazole) and eluted with elution buffer (20 mM Tris, pH 7.5, 500 mM NaCl, 500 mM imidazole). The eluted protein was further purified by using a 5 ml HiTrap Q HP column (Cytiva) equilibrated with 85% buffer A (20 mM HEPES, pH 7.5 and 5 mM DTT) and 15% buffer B (20mM HEPES, pH 7.5, 1 M NaCl, 5 mM DTT). The protein was eluted with a gradient from 15% to 60% buffer B. Fractions of interest were concentrated and stored at −80° C.

In Vitro Reconstitution and SUMO-Tag Cleavage for shRNP

2 molar equivalents of SmD3/SmB, SmG/SmE, and SUMO-SmD1/SmD2-EGF complexes were mixed with 1 molar equivalent of shRNA (synthesized by Dharmacon, Horizon Discovery) in 500 μL reconstitution buffer (20 mM HEPES, pH 7.5, 750 mM NaCl, 5 mM ethylenediaminetetraaceticacid (EDTA), and 5 mM DTT). Prior to mixing, the shRNA was reannealed by heating to 90° C. for 5 min and snap-cooling on ice for 10 min. The RNA/Protein mixture was incubated at 30° C. for 30 min, followed by 37° C. for 15 min and then cooled on ice for 10 min. The SUMO-tag on SmD1 was then removed by incubating with ULP1 protease overnight at 4° C. The reconstituted complex was purified by size exclusion chromatography (Superdex® 200 increase 10/300 GL column, GE Healthcare) with a buffer containing 20 mM HEPES, pH 7.5, 150 mM NaCl, 10 mM EDTA, and 5 mM DTT. Relevant fractions were pooled, concentrated and store at −80° C.

The shRNA sequences used for shRNP were as follows:

1) shRNA targeting KRAS
(SEQ ID NO: 27)
5-6-FAM-CAA UUU UUG ACC UUG ACG AUA CAG CUA AUU
CCU CGA GGA AUU AGC UGU AUC GUC AAG G-3’
2)shRNA targeting egfp
(SEQ ID NO: 28)
5-DY547-CAA UUU UUG AGC AAG CUG ACC CUG AAG UUC
ACU CGA GUG AAC UUC AGG GUC AGC UUG C-3’

In Vitro Reconstitution for shRNP, siRNP, shRNP-IGF and siRNP-IGF

Equimolar amounts of SmD1/SmD2, His-SmD3/SmB₁₋₉₅ or SmB₁₋₉₅, IGF-SmG-His/SmE/SmF complexes and shRNA or siRNA were mixed in 750 μL reconstitution buffer (20 mM HEPES, pH 7.5, 500 mM NaCl, 5 mM ethylenediaminetetraacetic acid (EDTA), and 5 mM DTT). Prior to mixing, the shRNA or siRNA was reannealed by heating to 90° C. for 5 min and snap-cooling on ice for 10 min. The RNA/Protein mixture was incubated at 30° C. for 30 min, followed by 37° C. for 15 min and then cooled on ice for 10 min. The reconstituted complex was purified by size exclusion chromatography (Superdex® 200 increase 10/300 GL column, Cytiva) with a buffer containing 20 mM HEPES, pH 7.5, 150 mM NaCl, 10 mM EDTA, and 5 mM DTT. Relevant Fractions were pooled, concentrated and store at −80° C.

In vitro reconstitution for shRNP containing EEPs (shRNP-IGF-GALA3-HSE, shRNP-IGF, shRNP-IGF-EGF, shRNP-EGF, siRNP-IGF-HSE, siRNP-EGF-H5E)

1.5 molar equivalents of SmD1/SmD2, His-SmD3/SmB₁₋₉₅ or SmB₁₋₉₅-GALA3 or SmB₁₋₉₅-H5E, IGF-SmG-His/SmE/H5E-SmF or SmG-His/SmE-EGF/SmF complexes and 1 molar equivalent of shRNA or siRNA were mixed in 750 μL reconstitution buffer (20 mM HEPES, pH 7.5, 500 mM NaCl, 5 mM EDTA, and 5 mM DTT). Prior to mixing, the shRNA or siRNA was reannealed by heating to 90° C. for 5 min and snap-cooling on ice for 10 min. The RNA/Protein mixture was incubated at 30° C. for 30 min, followed by 37° C. for 15 min and then cooled on ice for 10 min. The reconstituted complex was purified by size exclusion chromatography (Superdex® 200 increase 10/300 GL column, Cytiva) with a buffer containing 20 mM HEPES, pH 7.5, 500 mM NaCl, 10 mM EDTA, and 5 mM DTT. Relevant Fractions were pooled, concentrated and store at −80° C.

### Cell Line A549 and Culture Medium

Human lung adenocarcinoma cell line A549 was maintained in RPMI-1640 medium (Gibco, Life Technologies) with 2 g/L sodium bicarbonate(Sigma-Aldrich) and supplemented with penicillin and streptomycin(Gibco, Life Technologies), and 10% fetal bovine serum (Gibco, Life Technologies) at 37° C., 5% CO₂.

### Cell Line 293T-eGFP and Culture Medium

Human embryonic kidney cell line 293T with endogenous enhanced green fluorescent protein expression (293T-eGFP) was maintained in DMEM, high glucose (Gibco, Life Technologies) with 3.7 g/L sodium bicarbonate and supplemented with penicillin and streptomycin, 5% fetal bovine and 5% Newborn calf serum(Gibco, Life Technologies) at 37° C., 5% CO₂.

Gene Expression Analysis for shRNP

A549 cells were seeded in 12-well flat-bottomed tissue culture plate (Falcon BD) and incubated overnight. Culture medium was replenished with medium supplemented with a final concentration 123 nM (each) protein subcomplex (His-SUMO-SmD1/SmD2-EGF, SmD3/SmB₁₋₉₅, SmG-His/SmE), 123 nM shRNP, and buffer negative control and incubated at 37° C., 5% CO₂. After 4 days, total RNA was isolated using RNAzol RT (Molecular Research Center) and reverse transcribed into cDNA using M-MLV Reverse Transcriptase (Promega) according to the manufacturer's protocol. Quantitative real-time PCR reactions were performed using synthesized cDNA with TB Green Premix Ex Taq II (Takara) on a StepOnePlus Real-Time PCR instrument (Applied Biosystems). The sequences of the primers used in the RT-PCR were as follows: KRAS 1 forward primer (SEQ ID NO: 29) and reverse primer (SEQ ID NO: 30), KRAS 2 forward primer (SEQ ID NO: 31) and reverse primer (SEQ ID NO: 32), GAPDH forward primer (SEQ ID NO: 33) and reverse primer (SEQ ID NO: 34). Relative levels of KRAS expression were calculated using the ΔΔCT method with GAPDH as the normalization control.

Gene Expression Analysis for Cell Treatment with shRNP-H5E and shRNP-GALA3-H5E

A549 cells were seeded in 12-well flat-bottomed tissue culture plate (Falcon BD) and incubated overnight. The culture media were replenished with medium supplemented with protein control (100 nM of each subcomplex: SmD1/SmD2, His-SmD3/SmB₁₋₉₅ or His-SmD3/SmB₁₋₉₅-GALA3, IGF-SmG-His/SmE/H5E-SmF), 100 nM shRNA (targeting KRAS), 100 nM shRNP, and buffer control. The cells were then incubated at 37° C., 5% CO₂. After 2 days, total RNA was isolated using RNAzol RT (Molecular Research Center) and reverse transcribed into cDNA using M-MLV Reverse Transcriptase (Promega) according to the manufacturer's protocol. Duplicate quantitative real-time PCR reactions were performed using synthesized cDNA with iTaq Universal SYBR Green Supermix (Bio-Rad) on a StepOnePlus Real-Time PCR instrument (Applied Biosystems). The sequences of the primers used in the RT-PCR were as follows: KRAS 2 forward (SEQ ID NO: 31) and reverse (SEQ ID NO: 32), GAPDH forward (SEQ ID NO: 33) and reverse (SEQ ID NO: 34). Relative levels of KRAS expression were calculated using the ΔΔCT method with GAPDH as the normalization control.

Gene Expression Assay for Cell Treatment with siRNPs

HCT116 cell was purchased from American Type Culture Collection (ATCC). HCT116 cells were seeded in 24-well flat-bottomed tissue culture plate (JET Biofil) and incubated overnight. The culture media were replenished with medium supplemented with buffer, RNA (100-1000 nM), protein (100-1000 nM) controls and siRNP (100-1000 nM) respectively. The cells were then incubated at 37° C., 5% CO₂. The cells were lysed in TRIzol (Invitrogen) after 48 h and total RNA was extracted using the Direct-zol RNA kit (Zymo Research) following the manufacturer's protocol. The extracted RNA was reverse transcribed into cDNA using PrimeScript RT Reagent Kit (Takara). Duplicate quantitative real-time PCR was then performed on the synthesized cDNA using the iTaq Universal SYBR Green Supermix (Bio-Rad) on a ViiA 7 Real-Time PCR system (Applied Biosystems). The sequences of the primers used in the RT-PCR for KRAS and GAPDH were the same as above (SEQ ID NOS: 31-34). Relative levels of KRAS expression were calculated using the ΔΔCT method with GAPDH as the normalization control.

Human coronavirus strain 229E (HCOV-229E) was purchased from ATCC, and propagated in MRCS cells. After 5 days of inoculation, the infected media were collected and centrifuged at 4000 rpm at 4° C. for 10 min. The supernatant was aliquoted and stored at −80° C. before use. For testing the effectiveness of the siRNP complexes, VeroE6 cells (JCRB Cell Bank) were first infected by HCOV-229E by replenishing the media with 250 μL of infected media from the MRCS cells, and incubated for 2 h. The infected media were then discarded, the cells washed with PBS, and 200 μL media supplemented with 500 nM of siRNP(S)-IGF-H5E, or siRNP(E)-IGF-H5E, or buffer were added to the cells. The uninfected (mock) cells were washed with PBS, and media supplemented with 500 nM of siRNP(S)-IGF-H5E, or siRNP(E)-IGF-H5E, or buffer were added to the cells. After 48 h post-infection, the cells were harvested for total RNA using RNAiso Plus (Takara), subjected to reverse transcription (Takara PrimeScript RT Reagent Kit), and quantitative (q)PCR to examine S and E gene expression. The sequences of the primers were:

SPIKE
forward
(SEQ ID NO: 49)
(5’-ACCTATCGTAGTTGATTGCTC-3’)
and
reverse
(SEQ ID NO: 50)
(5’-AGCATCTCACTAACATCTGC-3’);
ENV
forward
(SEQ ID NO: 51)
(5’-TGGTTGTTAATGTACTACTCTGGTG-3')
and
reverse
(SEQ ID NO: 52)
(5’-ACATATGGCAAGTGAAACAAAGC-3');
ACTB
forward
(SEQ ID NO: 53)
(5’-ACTCTTCCAGCCTTCCTTCC-3’)
and
reverse
SEQ ID NO: 54)
(5’-CGTACAGGTCTTTGCGGATG-3’).

Relative levels of S and E expression were calculated using the ΔΔCT method with ACTB gene encoding for beta-actin as the normalization control.

### Confocal Microscopy

For the Investigations of shRNP_{KRAS} on A549 Cells

A549 cells were seeded in glass-bottomed tissue culture dish and incubated overnight. Culture medium was replenished with a 123 nM each protein subcomplex-,123 nM shRNA-, 123 nM shRNP- and buffer-added medium and incubated for 2 days, while another with a buffer only as negative control. Cells were co-stained with Hoechst 33342 nucleic acid stain (0.3 ug/ml, Invitrogen) before imaging. Images of fluorescence-labelled cells were captured with a Nikon Eclipse Ti2 confocal laser-scanning microscope. Hoechst 33342 signal was captured in the blue (λₑₓ=400 nm, λₑₘ=410-480 nm) channel and 6-FAM signal was captured in the green (λₑₓ=491 nm, λₑₘ=500-550 nm) channel.

For the Investigations of shRNP_{KRAS} Containing Endosomal Escape Peptide on A549 Cells

A549 cells were seeded in glass-bottomed tissue culture dish and incubated overnight. The culture media were replenished with media supplemented with protein control (100 nM of each subcomplex: SmD1/SmD2, His-SmD3/SmB₁₋₉₅ or His-SmD3/SmB₁₋₉₅-GALA3, IGF-SmG-His/SmE/H5E-SmF), 100 nM shRNA targeting KRAS, 100 nM shRNP and buffer control and incubated for 2 days. The cells were costained with Hoechst 33342 nucleic acid stain (0.3 ug/ml, Invitrogen) before imaging. Images of fluorescence-labelled cells were captured with a Nikon Eclipse Ti2 confocal laser-scanning microscope. Hoechst 33342 signal was captured in the blue (λₑₓ=400 nm) channel and 6-FAM signal was captured in the green (λₑₓ=491 nm) channel.

For the Investigations of shRNP, on 293T-eGFP Cells

293T-eGFP cells were seeded in glass-bottomed culture dish and incubated overnight. Culture medium was replenished with 100 nM each protein subcomplex-,100 nM shRNA-, 100 nM shRNP- and buffer-added medium and incubated for 2 days, with buffer as negative control. Cells were co-stained with Hoechst 33342 nucleic acid stain before imaging. Images of fluorescence-labelled cells were captured with a Nikon Eclipse Ti2 confocal laser-scanning microscope. Hoechst 33342 signal was captured in the blue (λₑₓ=400 nm, λₑm=410-480 nm) channel. eGFP signal was captured in the green (λₑₓ=491 nm, λₑₘ=510-540 nm) channel. DY547 signal was captured in the red (λₑₓ=561 nm, λₑₘ=570-600 nm) channel.

### Cell Viability Assay

A549 cells were seeded in 96-well flat-bottomed tissue culture plate (Nunc) and incubated overnight. Culture medium was replenished with medium supplemented with a final concentration 123 nM and 246 nM (each) protein subcomplex (His-SUMO-SmD1/SmD2-EGF, SmD3/SmB₁₋₉₅, SmG-His/SmE), 123 nM and 246 nM shRNP, and buffer negative control and incubated at 37° C., 5% CO₂. After 3 days, culture medium was replaced by medium added with MTT reagent (3-(4, 5-dimethylthiazol-2, 5-diphenyltetrazolium bromide, 0.5 mg/ml, Sigma-Aldrich) and incubated for 2 h. The media was then removed and the formazan salt crystals formed were dissolved with 100 μL dimethyl sulfoxide (RCI Labscan) with 30 min shaking. Absorbance was measured at 540 nm wavelength with reference at 690 nm wavelength on a Biotek ELx800 microplate reader (Biotek). Cell viability (%) was calculated according to the equation:

Cell Viability(%)=(OD_{protein or shRNP-complex}/OD_{buffer})×100%

MTT (3-[4,5-dimethylthiazol-2-yl]-2,5-diphenylterazolium bromide) assay was performed to determine the cytotoxicity of siRNP on HCT116 cells. HCT116 cells (3×10³/well in 100 μl DMEM) were seeded in 96-well tissue culture plate (SPL) and allowed to attach overnight at 37° C. The culture media were replenished with media supplemented with buffer, protein (100-1000 nM), RNA (100-1000 nM) control and siRNP (100-1000 nM) respectively. After 72 h of treatment, 10 μl of MTT solution (5 mg/ml) was added to each well and incubated at 37° C., 5% CO₂ for 2 hours. The medium was then aspirated, and 100 pi of DMSO was added to dissolve the formed formazan crystals. The absorbance was measured at 570 nm and referenced at 630 nm by a microplate spectrophotometer (BD Biosciences, USA). The assays were carried out in duplicate.

### Internalization Assay

A549 cells (3×10⁴/well in 500 μl RPMI 1640) were seeded in 24-well tissue culture plate (SPL) and allowed to attach overnight at 37° C. The culture media were replenished with media supplemented with 100 nM Dy547-shRNA, Dy547-shRNP-EGF-HSE, Dy547-shRNP-IGF-HSE, Dy547-shRNP-IGF/EGF-H5E respectively, and buffer as negative control. After incubation of 1 h, 2 h, and 4 h, the cells were trypsinized, centrifuged and washed with PBS for 3 times. The cell pellets were resuspended with 500 μL, PBS and analyzed by BD ACCURI™ C6 Plus Flow Cytometer (BD Biosciences) utilizing 10,000 events. The mean fluorescence intensities (MFI) were calculated by FlowJo (Version 10).

### INDUSTRIAL APPLICATION

The present invention serves as a platform technology to deliver RNA therapeutics into cells. As RNA therapeutics has immense potential applications in biotechnology, for example gene silencing for chronic disease and cancer treatment, RNA-based inhibitors as well as RNA-based vaccines, this invention provides a system for delivery of RNA molecules for biomedical purposes. The modular protein-based system described in this invention allows for customization of protein modules to achieve specificity in cell-targeting, thus having the ability to be optimized for treating different diseases. Examples of types of diseases that could adopt this technology for treatment include cancer, neurodegenerative diseases and viral infection. This invention could also be applied to RNA delivery for research purposes, be used in cell lines and in vivo studies.

What we claim:

1. An RNA therapeutic delivery agent comprising a modified small nuclear ribonucleoprotein (snRNP) complex for delivering a therapeutic RNA to a biological cell, said modified snRNP complex comprising a core, the core comprising:
one or more RNA molecules;
one or more Sm proteins, or one or more LSm proteins, or any combination of Sm and LSm proteins, or any variants thereof; and
a Sm binding sequence,
wherein the Sm binding sequence is attached to the therapeutic RNA,
wherein the therapeutic RNA is bound to at least one of the Sm proteins or at least one of the LSm proteins, or any combination or variant thereof, of the modified snRNP complex,
wherein at least one cell receptor ligand is attached to at least one of the Sm proteins or at least one of the LSm proteins, or any combination or variant thereof, of the modified snRNP complex,
wherein at least one endosomal escape peptide is attached to at least one of the Sm proteins or at least one of the LSm proteins, or any combination or variants thereof, of the modified snRNP complex.

2. The agent of claim 1, wherein the one or more Sm proteins comprise SmD3, SmF, SmB, SmG, SmE, SmD1 and SmD2 of SEQ ID NOs: 1-7, respectively, or the variants thereof including SmB′ of SEQ ID NO: 9, SmD1′ of SEQ ID NO: 10, IGF-SmG of SEQ ID NO: 39, SmB₁₋₉₅-GALAS of SEQ ID NO: 41, and H5E-SmF of SEQ ID NO: 42.

3. The agent of claim 1, wherein the LSm proteins comprise LSm1, LSm2, LSm3, LSm4, LSm5, LSm6, LSm7, LSm8, LSm10, and LSm11 of SEQ ID NOs: 11-20, respectively.

4. The agent of claim 1, wherein the cell receptor ligand is for receptor-mediated endocytosis comprising epidermal growth factor (EGF), or insulin-like growth factor (IGF), and any family members thereof, and wherein the EGF or any family members thereof is/are attached to any of the Sm proteins, or the variants thereof; the IGF or any family members thereof is/are attached to any of the Sm proteins, or the variants thereof.

5. The agent of claim 1, wherein the therapeutic RNA is incorporated into the short-hairpin ribonucleoprotein complex (shRNP) comprising an shRNA of SEQ ID NO: 27 or SEQ ID NO: 36 attached with a 6-FAM fluorescent label at 5′-end thereof for targeting KRAS, an shRNA of SEQ ID NO: 28 attached with a DY547 dye at 5′-end thereof for targeting egfp, or a small-interfering RNA (siRNA) of SEQ ID Nos: 37 and 38 for targeting KRAS, a pair of siRNAs of SEQ ID NOs: 45 and 46 for targeting a spike protein of a coronavirus, or a pair of siRNAs of SEQ ID NOs: 47 and 48 for targeting an envelope protein of the coronavirus.

6. The agent of claim 1, wherein the Sm binding sequence is attached to the therapeutic RNA at either 3′-end or 5′-end thereof.

7. The agent of claim 1, wherein the cell receptor ligand is attached to N-terminus, C-terminus, or within a loop between β strands 3 and 4 of any one of the Sm proteins or any one of the LSm proteins, or any variants thereof.

8. The agent of claim 1, wherein the one or more RNA molecules comprise small nuclear RNA.

9. The agent of claim 1, wherein the Sm binding sequence is one of the following nucleotide sequences:
SEQ ID NO: 21:
5’-AAUUUGUGG-3’;
SEQ ID NO: 22:
5’-GAUUUUUGG-3’;
SEQ ID NO: 23:
5’-AAUUUUUGA-3’;
SEQ ID NO: 24:
5’-AAUUUUUUG-3’;
SEQ ID NO: 25:
5’-UUUU-3’;
SEQ ID NO: 26:
5’-AAUUUGUCUAG-3’

10. The agent of claim 1, wherein the at least one endosomal escape peptide comprises H5E or GALA3.

11. A modified small nuclear ribonucleoprotein (snRNP) complex for gene silencing in a cell, said snRNP complex comprising a core, the core comprising:
one or more RNA molecules;
one or more Sm proteins, or one or more LSm proteins, or any combination of the Sm and LSm proteins, or any variants thereof; and
a Sm binding sequence,
wherein the Sm binding sequence is attached to a therapeutic RNA including shRNA or siRNA to be delivered to the cell, and
wherein the shRNA or siRNA is bound to at least one of the Sm proteins or at least one of the LSm proteins, or any combination or variants thereof, of the modified snRNP complex,
wherein at least one cell receptor ligand is attached to at least one of the Sm proteins or at least one of the LSm proteins, or any combination or variants thereof, of the modified snRNP complex, and
wherein at least one endosomal escape peptide is attached to at least one of the Sm proteins or at least one of the LSm proteins, or any combination or variants thereof, of the modified snRNP complex.

12. The complex of claim 11, wherein the one or more Sm proteins comprise SmD3, SmF, SmB, SmG, SmE, SmD1 and SmD2 of SEQ ID Nos: 1-7, respectively, or the variants thereof including SmB′ of SEQ ID NO: 9, SmD1′ of SEQ ID NO: 10, IGF-SmG of SEQ ID NO: 39, SmB₁₋₉₅-GALA3 of SEQ ID NO: 41, and H5E-SmF of SEQ ID NO: 42.

13. The complex of claim 11, wherein the LSm proteins comprise LSm1, LSm2, LSm3, LSm4, LSm5, LSm6, LSm7, LSm8, LSm10, and LSm11 of SEQ ID Nos: 11-20, respectively.

14. The complex of claim 11, wherein the at least one cell receptor ligand comprises an epidermal growth factor (EGF), or insulin-like growth factor (IGF), and any family members thereof, and wherein the EGF or any family members thereof is/are attached to any of the Sm proteins, or the variants thereof; the IGF or any family members thereof is/are attached to any of the Sm proteins, or the variants thereof.

15. The complex of claim 11, wherein the shRNP comprises an shRNA of SEQ ID NO: 27 or SEQ ID NO: 36 attached with a 6-FAM fluorescent label at 5′-end thereof for targeting KRAS, an shRNA of SEQ ID NO: 28 attached with a DY547 dye at 5′-end thereof for targeting egfp, an siRNA of SEQ ID NO: 37 or 38 for targeting KRAS, a pair of siRNAs of SEQ ID NOs: 45 and 46 for targeting a spike protein of a coronavirus, or a pair of siRNAs of SEQ ID NOs: 47 and 48 for targeting an envelope protein of the coronavirus.

16. The complex of claim 11, wherein the Sm binding sequence is attached to the shRNA or siRNA at either 3′-end or 5′-end thereof.

17. The complex of claim 11, wherein the cell receptor ligand is attached to N-terminus, C-terminus, or within a loop between P strands 3 and 4 of any one of the Sm proteins or any one of the Sm proteins, or any variants thereof.

18. The complex of claim 11, wherein the one or more RNA molecules comprise small nuclear RNA.

19. The complex of claim 11, wherein the Sm binding sequence is one of the following nucleotide sequences:
SEQ ID NO: 21:
5’-AAUUUGUGG-3’;
SEQ ID NO: 22:
5’-GAUUUUUGG-3’;
SEQ ID NO: 23:
5’-AAUUUUUGA-3’;
SEQ ID NO: 24:
5’-AAUUUUUUG-3’;
SEQ ID NO: 25:
5’-UUUU-3’;
SEQ ID NO: 26:
5’-AAUUUGUCUAG-3’

20. The complex of claim 11, wherein the at least one endosomal escape peptide comprises H5E or GALA3.

21. A method for treating diseases in a subject comprising administering the agent of claim 1 to the subject in need thereof.

22. A method for treating diseases in a subject comprising administering the modified snRNP complex of claim 11 to the subject in need thereof.
